(19) United States
(12) Patent Application Publication
(10) Pub. No.: US 2011/0124667 A1
(43) Pub. Date: May 26, 2011

(54) METHODS FOR THE TREATMENT OF IRRITABLE BOWEL SYNDROME

(76) Inventor: Philip Manton BROWN, The Woodlands, TX (US)

(21) Appl. No.: 12/949,978

(22) Filed: Nov. 19, 2010

Related U.S. Application Data
(60) Provisional application No. 61/263,565, filed on Nov. 23, 2009.

(51) Int. Cl. A61K 31/505; A61P 1/00

(52) U.S. Cl. 514/272

(57) ABSTRACT

Methods of treating irritable bowel syndrome (IBS) are disclosed. Assays and kits useful in the treatment of IBS are also disclosed.

This application claims priority to U.S. provisional patent application No. 61/263,565, filed Nov. 23, 2009, the entirety of which is incorporated herein by reference.

### 1. FIELD OF THE INVENTION

This invention relates to methods of treating irritable bowel syndrome, and assays and kits useful therein.

### 2. BACKGROUND

Irritable bowel syndrome (IBS) is a common functional gastrointestinal (GI) disorder, having an estimated prevalence rate of 10-15 percent in industrialized countries. Maxion-Bergemann, S., et al., Pharmacoeconomics 24(1):21-37, 21 (2006). The disorder is characterized by abdominal pain or discomfort associated with a change in bowel habits such as constipation (IBS-C), diarrhea (IBS-D), or alternating between the two conditions (IBS-A). A standardized approach to evaluating and diagnosing IBS patients is aided by criteria established over the past 30 years, including what are known as Manning criteria, Rome I criteria, Rome II criteria, and Rome III criteria. See, e.g., Rome III: The Functional Gastrointestinal Disorders, Drossman, D. A., ed. (3^{rd} edition, January 2006), Appendix A; Drossman, D. A., Gastroenterology 20(5):1377-1390 (2006).

The various criteria used to diagnose IBS are based on symptoms, such as abdominal discomfort. Although it is desirable to correlate IBS with one or more biomarkers, the task is daunting: more than 600 biological pathways have been associated with IBS and other GI diseases. Lembo, A. J., et al., Aliment. Pharmacol. Ther. 29:834-842, 835 (2009).

One pathway that has been associated with GI functional disorders is the serotenergic pathway. Serotonin—chemically named 5-hydroxytriptamine (5-HT))—is a neurotransmitter that has both central and peripheral effects. In the periphery, it reportedly plays a role in vascular tone, gut motility, primary hemostasis, and cell-mediated immune responses. Walther, D. J., et al., Science 299:76 (2003). Serotonin is synthesized in vivo from the amino acid L-tryptophan with the aid of tryptophan hydroxylase (TPH), the enzyme that catalyzes the process' rate limiting step. Id. One of the two known isoforms of the enzyme, TPH1, is expressed primarily in the enterochromaffin cells of the GI tract, from which most of the body's peripheral serotonin is derived. Id.

It has been reported that some IBS-D patients exhibit increased levels of blood 5-HT and its metabolite 5-hydroxyindoleacetic acid (5-HIAA) as compared to normal, disease-free subjects. See Atkinson, W. et al., Gastroenterology 130:34-43, 34 (2006). It has also been reported that some IBS patients exhibit different responses to changes in tryptophan load than normal subjects. See Shufflebotham, J., et al., Am. J. Gastroenterol 101:2582-2587 (2006). However, the set of biomarkers that is reportedly the most predictive of IBS does not include 5-HT or 5-HIAA. Lembo at 836.

### 3. SUMMARY OF THE INVENTION

This invention encompasses methods of treating and managing irritable bowel syndrome (IBS). For example, one embodiment of the invention encompasses a method of treating or managing non-constipating irritable bowel syndrome (IBS), which comprises administering to a patient suffering from non-constipating IBS an amount of a tryptophan hydroxylase (TPH) inhibitor sufficient to lower the patient's blood 5-HT level by at least about 10 ng/mL compared to baseline.

Another embodiment encompasses a method of treating or managing non-constipating IBS, which comprises administering to a patient suffering from non-constipating IBS a therapeutically effective amount of a TPH inhibitor, during which treatment or management the patient exhibits a lowering in blood 5-HT level of at least about 10 ng/mL compared to baseline.

Another embodiment encompasses a method of treating or managing non-constipating IBS, which comprises administering to a patient suffering from non-constipating IBS an amount of a TPH inhibitor sufficient to lower the patient's 5-HIAA level by at least about 10 percent compared to baseline.

Another embodiment encompasses a method of treating or managing non-constipating IBS, which comprises administering to a patient suffering from non-constipating IBS a therapeutically effective amount of a TPH inhibitor, during which treatment or management the patient exhibits a lowering in blood or urinary 5-HIAA level of at least about 10 percent compared to baseline.

This invention also encompasses methods and kits for determining whether an IBS patient is likely to respond to TPH inhibitor therapy. For example, one embodiment of the invention encompasses a method of determining if a patient suffering from IBS will be responsive to TPH inhibitor therapy. Another encompasses a kit for determining whether a patient suffering from IBS will be responsive to TPH inhibitor therapy.

### 4. BRIEF DESCRIPTION OF THE FIGURES

Aspects of the invention can be understood from the appended figures:

FIG. 1 shows the effect of an orally available TPH inhibitor on the urinary 5-HIAA levels of 40 normal, healthy volunteers in a phase 1b multi-dose clinical study.

FIG. 2 provides results obtained from a phase 2a study of the TPH inhibitor in non-constipating IBS patients. In particular, it shows that patients randomized to the high dose arm showed significant improvement versus placebo in weekly global assessment over the 28 day treatment period. The day the final dose was administered is indicated by an arrow.

FIG. 3 also provides results from the phase 2a study, wherein patients randomized to the high dose arm showed significant improvement in stool consistency (Bristol Stool Scale).

FIG. 4 provides additional results from the phase 2a study, wherein patients exhibited a reduction in 24-hour urinary 5-HIAA levels upon treatment with the TPH inhibitor.

### 5. DETAILED DESCRIPTION

This invention is based, in part, on discoveries made during the first known human clinical trials of a TPH inhibitor in patients suffering from IBS.

### 5.1. DEFINITIONS

Unless otherwise indicated, the term “alkenyl” means a straight chain, branched and/or cyclic hydrocarbon having from 2 to 20 (e.g., 2 to 10 or 2 to 6) carbon atoms, and including at least one carbon-carbon double bond. Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl.

Unless otherwise indicated, the term “alkoxy” means an —O-alkyl group. Examples of alkoxy groups include, but are not limited to, —OCH₃, —OCH₂CH₃, —O(CH₂)₂CH₃, —O(CH₂)₃CH₃, —O(CH₂)₄—CH₃, and —O(CH₂)₅CH₃.

Unless otherwise indicated, the term “alkyl” means a straight chain, branched and/or cyclic (“cycloalkyl”) hydrocarbon having from 1 to 20 (e.g., 1 to 10 or 1 to 4) carbon atoms. Alkyl moieties having from 1 to 4 carbons are referred to as “lower alkyl.” Examples of alkyl groups include, but are not limited to, methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). The term “alkyl” includes saturated hydrocarbons as well as alkenyl and alkynyl moieties.

Unless otherwise indicated, the term “alkylaryl” or “alkyl-aryl” means an alkyl moiety bound to an aryl moiety.

Unless otherwise indicated, the term “alkylheteroaryl” or “alkyl-heteroaryl” means an alkyl moiety bound to a heteroaryl moiety.

Unless otherwise indicated, the term “alkylheterocycle” or “alkyl-heterocycle” means an alkyl moiety bound to a heterocycle moiety.

Unless otherwise indicated, the term “alkynyl” means a straight chain, branched or cyclic hydrocarbon having from 2 to 20 (e.g., 2 to 20 or 2 to 6) carbon atoms, and including at least one carbon-carbon triple bond. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl.

Unless otherwise indicated, the term “aryl” means an aromatic ring or an aromatic or partially aromatic ring system composed of carbon and hydrogen atoms. An aryl moiety may comprise multiple rings bound or fused together. Examples of aryl moieties include, but are not limited to, anthracenyl, azulenyl, biphenyl, fluorenyl, indan, indenyl, naphthyl, phenanthrenyl, phenyl, 1,2,3,4-tetrahydro-naphthalene, and tolyl.

Unless otherwise indicated, the term “arylalkyl” or “aryl-alkyl” means an aryl moiety bound to an alkyl moiety.

Unless otherwise indicated, the terms “halogen” and “halo” encompass fluorine, chlorine, bromine, and iodine.

Unless otherwise indicated, the term “heteroalkyl” refers to an alkyl moiety (e.g., linear, branched or cyclic) in which at least one of its carbon atoms has been replaced with a heteroatom (e.g., N, O or S).

Unless otherwise indicated, the term “heteroalkylaryl” or “heteroalkyl-aryl” refers to a heteroalkyl moiety bound to an alkyl moiety.

Unless otherwise indicated, the term “heteroalkylheterocycle” or “heteroalkyl-heterocycle” refers to a heteroalkyl moiety bound to heterocycle moiety.

Unless otherwise indicated, the term “heteroaryl” means an aryl moiety wherein at least one of its carbon atoms has been replaced with a heteroatom (e.g., N, O or S). Examples include, but are not limited to, acridinyl, benzimidazolyl, benzofuranyl, benzoisothiazolyl, benzoisoxazolyl, benzoquinazolinyl, benzothiazolyl, benzoxazolyl, furyl, imidazolyl, indolyl, isothiazolyl, isoxazolyl, oxadiazolyl, oxazolyl, phthalazinyl, pyrazinyl, pyrazolyl, pyridazinyl, pyridyl, pyrimidinyl, pyrimidyl, pyrrolyl, quinazolinyl, quinolinyl, tetrazolyl, thiazolyl, and triazinyl.

Unless otherwise indicated, the term “heteroarylalkyl” or “heteroaryl-alkyl” means a heteroaryl moiety bound to an alkyl moiety.

Unless otherwise indicated, the term “heterocycle” refers to an aromatic, partially aromatic or non-aromatic monocyclic or polycyclic ring or ring system comprised of carbon, hydrogen and at least one heteroatom (e.g., N, O or S). A heterocycle may comprise multiple (i.e., two or more) rings fused or bound together. Heterocycles include heteroaryls. Examples include, but are not limited to, benzo[1,3]dioxolyl, 2,3-dihydro-benzo[1,4]dioxinyl, cinnolinyl, furanyl, hydantoinyl, morpholinyl, oxetanyl, oxiranyl, piperazinyl, piperidinyl, pyrrolidinonyl, pyrrolidinyl, tetrahydrofuranyl, tetrahydropyranyl, tetrahydropyridinyl, tetrahydropyrimidinyl, tetrahydrothiophenyl, tetrahydrothiopyranyl and valerolactamyl.

Unless otherwise indicated, the term “heterocyclealkyl” or “heterocycle-alkyl” refers to a heterocycle moiety bound to an alkyl moiety.

Unless otherwise indicated, the term “heterocycloalkyl” refers to a non-aromatic heterocycle.

Unless otherwise indicated, the term “heterocycloalkylalkyl” or “heterocycloalkyl-alkyl” refers to a heterocycloalkyl moiety bound to an alkyl moiety.

Unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

Unless otherwise indicated, the term “patient suffering from IBS” means a patient who exhibits symptoms of IBS (e.g., as defined in Rome III criteria). Symptoms of non-constipating IBS (i.e., IBS-D and/or IBS-A) include:
1. Recurrent abdominal pain or discomfort, defined as an uncomfortable sensation not described as pain at least 3 days/month and associated with two or more of the following characteristics:
   i. Improvement with defecation.
   ii. Onset associated with a change in frequency of stool.
   iii. Onset associated with a change in form (appearance) of stool.
2. Loose or watery stools for at least 25% of bowel movements and hard or lumpy stools for <25% of bowel movements (IBS-diarrhea), or loose or watery stools for at least 25% and hard or lumpy stool for at least 25% of bowel movements (IBS-mixed).

Unless otherwise indicated, the term “pharmaceutically acceptable salts” refers to salts prepared from pharmaceutically acceptable non-toxic acids or bases including inorganic acids and bases and organic acids and bases. Suitable pharmaceutically acceptable base addition salts include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art. See, e.g., Remington's Pharmaceutical Sciences, 18^{th} ed. (Mack Publishing, Easton Pa.: 1990) and Remington: The Science and Practice of Pharmacy, 19^{th} ed. (Mack Publishing, Easton Pa.: 1995).

Unless otherwise indicated, a “prophylactically effective amount” of a compound is an amount sufficient to prevent a disease or condition, or one or more symptoms associated with the disease or condition, or prevent its recurrence. A “prophylactically effective amount” of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

Unless otherwise indicated, the term “substituted,” when used to describe a chemical structure or moiety, refers to a derivative of that structure or moiety wherein one or more of its hydrogen atoms is substituted with a chemical moiety or functional group such as, but not limited to, alcohol, aldehyde, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (e.g. —C(O)NH-alkyl-, -alkylNHC(O)alkyl), amidinyl (e.g., —C(NH)NH-alkyl-, —C(NR)NH₂), amine (primary, secondary and tertiary such as alkylamino, arylamino, arylalkylamino), aroyl, aryl, aryloxy, azo, carbamoyl (e.g., —NHC(O)O-alkyl-, —OC(O)NH-alkyl), carbamyl (e.g., CONH₂, CONH-alkyl, CONH-aryl, CONH-arylalkyl), carbonyl, carboxyl, carboxylic acid, carboxylic acid anhydride, carboxylic acid chloride, cyano, ester, epoxide, ether (e.g., methoxy, ethoxy), guanidino, halo, haloalkyl (e.g., —CCl₃, —CF₃, —C(CF₃)₃), heteroalkyl, hemiacetal, imine (primary and secondary), isocyanate, isothiocyanate, ketone, nitrile, nitro, oxo, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) and urea (e.g., —NHCONH-alkyl-).

Unless otherwise indicated, a “therapeutically effective amount” of a compound is an amount sufficient to provide a therapeutic benefit in the treatment or management of a disease or condition, or to delay or minimize one or more symptoms associated with the disease or condition. A “therapeutically effective amount” of a compound means an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Unless otherwise indicated, the terms “treat,” “treating” and “treatment” contemplate an action that occurs while a patient is suffering from the specified disease or disorder, which reduces the severity of the disease or disorder, or retards or slows the progression of the disease or disorder.

Unless otherwise indicated, the term “include” has the same meaning as “include, but are not limited to,” and the term “includes” has the same meaning as “includes, but is not limited to.” Similarly, the term “such as” has the same meaning as the term “such as, but not limited to.”

Unless otherwise indicated, one or more adjectives immediately preceding a series of nouns is to be construed as applying to each of the nouns. For example, the phrase “optionally substituted alky, aryl, or heteroaryl” has the same meaning as “optionally substituted alky, optionally substituted aryl, or optionally substituted heteroaryl.”

It should be noted that a chemical moiety that forms part of a larger compound may be described herein using a name commonly accorded it when it exists as a single molecule or a name commonly accorded its radical. For example, the terms “pyridine” and “pyridyl” are accorded the same meaning when used to describe a moiety attached to other chemical moieties. Thus, the two phrases “XOH, wherein X is pyridyl” and “XOH, wherein X is pyridine” are accorded the same meaning, and encompass the compounds pyridin-2-ol, pyridin-3-ol and pyridin-4-ol.

It should also be noted that if the stereochemistry of a structure or a portion of a structure is not indicated with, for example, bold or dashed lines, the structure or the portion of the structure is to be interpreted as encompassing all stereoisomers of it. Moreover, any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit.

### 5.2. TPH INHIBITORS

Compounds useful as TPH inhibitors include those disclosed in U.S. Pat. Nos. 7,723,345; 7,553,840; and 7,709,493.

Particular compounds are of the formula:

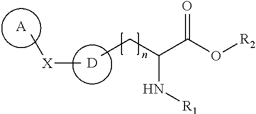

and pharmaceutically acceptable salts thereof, wherein: A is optionally substituted cycloalkyl, aryl, or heterocycle; X is a bond, —O—, —S—, —C(O)—, —C(R₄)═, ═C(R₄)—, —C(R₃R₄)—, —C(R₄)═C(R₄)—, —C≡C—, —N(R₅)—, —N(R₅)C(O)N(R₅)—, —C(R₃R₄)N(R₅)—, —N(R₅)C(R₃R₄)—, —ONC(R₃)—, —C(R₃)NO—, —C(R₃R₄)O—, —OC(R₃R₄)—, —S(O₂)—, —S(O₂)N(R₅)—, —N(R₅)S(O₂)—, —C(R₃R₄)S(O₂)—, or —S(O₂)C(R₃R₄)—; D is optionally substituted aryl or heterocycle; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; R₃ is hydrogen, alkoxy, amino, cyano, halogen, hydroxyl, or optionally substituted alkyl; R₄ is hydrogen, alkoxy, amino, cyano, halogen, hydroxyl, or optionally substituted alkyl or aryl; each R₅ is independently hydrogen or optionally substituted alkyl or aryl; and n is 0-3.

Particular compounds are of the formula:

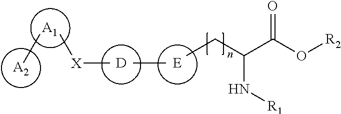

wherein: each of A₁ and A₂ is independently a monocyclic optionally substituted cycloalkyl, aryl, or heterocycle; and E is optionally substituted aryl or heterocycle. Some are of the formula:

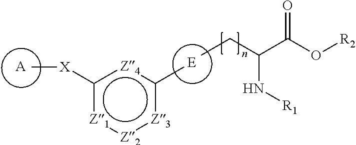

wherein: each of Z″₁, Z″₂, Z″₃, and Z″₄ is independently N or CR₁₀; each R₁₀ is independently amino, cyano, halogen, hydrogen, OR₁₁, SR₁₁, NR₁₂R₁₃, or optionally substituted alkyl, alkyl-aryl or alkyl-heterocycle; each R₁₁ is independently hydrogen or optionally substituted alkyl, alkyl-aryl or alkyl-heterocycle; each R₁₂ is independently hydrogen or optionally substituted alkyl, alkyl-aryl or alkyl-heterocycle; and each R₁₃ is independently hydrogen or optionally substituted alkyl, alkyl-aryl or alkyl-heterocycle.

Particular compounds are of the formula:

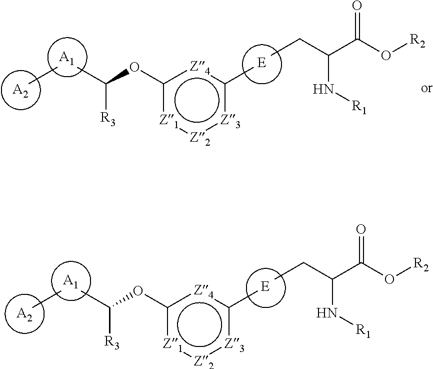

wherein, for example, R₃ is trifluoromethyl.

Some TPH inhibitors are compounds of the formula:

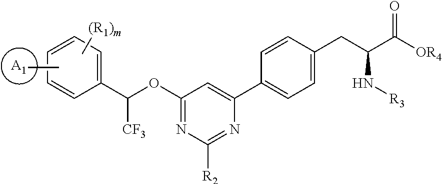

wherein: A₁ is optionally substituted heterocycle; each R₁ is independently halogen, hydrogen, C(O)R_{A}, OR_{A}, NR_{B}R_{C}, S(O₂)R_{A}, or optionally substituted alkyl, alkyl-aryl or alkyl-heterocycle; R₂ is independently halogen, hydrogen, C(O)R_{A}, OR_{A}, NR_{B}R_{C}, S(O₂)R_{A}, or optionally substituted alkyl, alkyl-aryl or alkyl-heterocycle; R₃ is hydrogen, C(O)R_{A}, C(O)OR_{A}, or optionally substituted alkyl, alkyl-aryl, alkyl-heterocycle, aryl, or heterocycle; R₄ is hydrogen or optionally substituted alkyl, alkyl-aryl, alkyl-heterocycle, aryl, or heterocycle; each R_{A} is independently hydrogen or optionally substituted alkyl, alkyl-aryl or alkyl-heterocycle; each R_{B} is independently hydrogen or optionally substituted alkyl, alkyl-aryl or alkyl-heterocycle; each R_{C} is independently hydrogen or optionally substituted alkyl, alkyl-aryl or alkyl-heterocycle; and m is 1-4.

Particular compounds are of the formula:

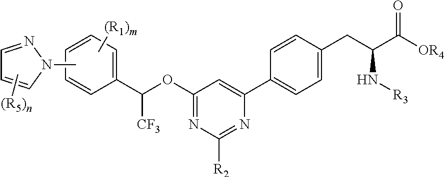

wherein: each R₅ is independently halogen, hydrogen, C(O)R_{A}, OR_{A}, NR_{B}R_{C}, S(O₂)R_{A}, or optionally substituted alkyl, alkyl-aryl or alkyl-heterocycle; and n is 1-3. Some are of the formula:

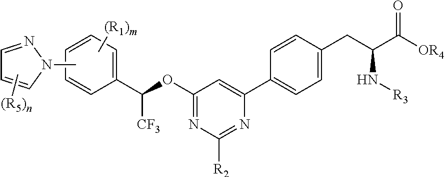

Specific TPH inhibitors include:
(S)-2-Amino-3-(4-(4-amino-6-((R)-1-(naphthalen-2-yl)ethylamino)-1,3,5-triazin-2-yl)phenyl)propanoic acid;
(S)-2-Amino-3-(4-(4-amino-6-((4′-methylbiphenyl-4-yl)methylamino)-1,3,5-triazin-2-yl)phenyl)propanoic acid;
(S)-2-Amino-3-(4-(4-morpholino-6-(naphthalen-2-ylmethylamino)-1,3,5-triazin-2-yl)phenyl)propanoic acid;
(2S)-2-Amino-3-(4-(2-amino-6-(2,2,2-trifluoro-1-(2-(trifluoromethyl)phenyl)ethoxy)pyrimidin-4-yl)phenyl)propanoic acid;
(2S)-2-Amino-3-(4-(2-amino-6-(2,2,2-trifluoro-1-p-tolylethoxy)pyrimidin-4-yl)phenyl)propanoic acid;
(2S)-2-Amino-3-(4-(2-amino-6-(1-cyclohexyl-2,2,2-trifluoroethoxy)pyrimidin-4-yl)phenyl)propanoic acid;
(S)-2-Amino-3-(4-(6-(2-fluorophenoxy)pyrimidin-4-yl)phenyl)propanoic acid;
(2S)-2-Amino-3-(4-(4-(3-(4-chlorophenyl)piperidin-1-yl)-1,3,5-triazin-2-yl)phenyl)propanoic acid;
(2S)-2-Amino-3-(4-(4-amino-6-(2,2,2-trifluoro-1-phenylethoxy)-1,3,5-triazin-2-yl)phenyl)propanoic acid;
(S)-2-Amino-3-(5-(4-amino-6-((R)-1-(naphthalen-2-yl)ethylamino)-1,3,5-triazin-2-yl)pyridin-2-yl)propanoic acid;
(S)-2-Amino-3-(3-(4-amino-6-((R)-1-(naphthalen-2-yl)ethylamino)-1,3,5-triazin-2-yl)-1H-pyrazol-1-yl)propanoic acid;
(S)-2-Amino-3-(4′-(3-(cyclopentyloxy)-4-methoxybenzylamino)biphenyl-4-yl)propanoic acid;
(S)-2-Amino-3-(4-(6-(3-(cyclopentyloxy)-4-methoxybenzylamino)pyrimidin-4-yl)phenyl)propanoic acid;
(S)-2-Amino-3-(4-(6-(3-(cyclopentyloxy)-4-methoxybenzylamino)pyrazin-2-yl)phenyl)propanoic acid;
(S)-2-Amino-3-(4-(5-((4′-methylbiphenyl-2-yl)methylamino)pyrazin-2-yl)phenyl)propanoic acid;
(2S)-2-Amino-3-(4-(6-(2,2,2-trifluoro-1-phenylethoxy)-pyrimidin-4-yl)phenyl)propanoic acid;
(2S)-2-Amino-3-(4-(6-(1-(3,4-difluorophenyl)-2,2,2-trifluoroethoxy)pyrimidin-4-yl)phenyl)propanoic acid;
(S)-2-Amino-3-(4-(5-(3-(cyclopentyloxy)-4-methoxybenzylamino)-pyrazin-2-yl)phenyl)propanoic acid;
(S)-2-Amino-3-(4-(5-((3-(cyclopentyloxy)-4-methoxybenzyl)-(methyl)amino)pyrazin-2-yl)phenyl)propanoic acid;
(S)-2-Amino-3-(4-(5-((1,3-dimethyl-1H-pyrazol-4-yl)methylamino)pyrazin-2-yl)phenyl)propanoic acid;
(S)-2-Amino-3-(4-(4-amino-6-((S)-1-(naphthalen-2-yl)ethylamino)-1,3,5-triazin-2-yloxy)phenyl)propanoic acid;
(S)-2-Amino-3-(4-(4-amino-6-((R)-1-(biphenyl-2-yl)-2,2,2-trifluoroethoxy)-1,3,5-triazin-2-yl)phenyl)propanoic acid;
(2S)-2-Amino-3-(4-(4-amino-6-(1-(6,8-difluoronaphthalen-2-yl)ethylamino)-1,3,5-triazin-2-yl)phenyl)propanoic acid;
(2S)-2-Amino-3-(4-(4-amino-6-(2,2,2-trifluoro-1-(3′-methylbiphenyl-2-yl)ethoxy)-1,3,5-triazin-2-yl)phenyl)propanoic acid;
(S)-2-Amino-3-(4-(5-(3,4-dimethoxyphenylcarbamoyl)-pyrazin-2-yl)phenyl)propanoic acid;
(S)-2-Amino-3-(4-(2-amino-6-(4-(2-(trifluoromethyl)phenyl)-piperidin-1-yl)pyrimidin-4-yl)phenyl)propanoic acid;
(S)-2-Amino-3-(4-(2-amino-6-((R)-1-(naphthalen-2-yl)ethylamino)pyrimidin-4-yl)phenyl)propanoic acid;
(S)-2-Amino-3-(4-(2-amino-6-(methyl((R)-1-(naphthalen-2-yl)ethyl)amino)pyrimidin-4-yl)phenyl)propanoic acid;
(S)-2-Amino-3-(4-(2-amino-6-((S)-2,2,2-trifluoro-1-(6-methoxynaphthalen-2-yl)ethoxy)pyrimidin-4-yl)phenyl)propanoic acid;
(S)-2-Amino-3-(4-(5-(biphenyl-4-ylmethylamino)pyrazin-2-yl)phenyl)propanoic acid;
(S)-2-Amino-3-(4-(5-(naphthalen-2-ylmethylamino)pyrazin-2-yl)phenyl)propanoic acid;
(S)-2-(Tert-butoxycarbonylamino)-3-(4-(5-(naphthalen-2-ylmethylamino)pyrazin-2-yl)phenyl)propanoic acid;
(S)-2-Morpholinoethyl 2-amino-3-(4-(5-(naphthalen-2-ylmethylamino)pyrazin-2-yl)phenyl)propanoate;
(2S)-2-Amino-3-(4-(2-amino-6-(2,2,2-trifluoro-1-(3′-fluorobiphenyl-4-yl)ethoxy)pyrimidin-4-yl)phenyl)propanoic acid;
(S)-2-Amino-3-(4-(2-amino-6-(benzylthio)pyrimidin-4-yl)phenyl)propanoic acid;
(S)-2-Amino-3-(4-(2-amino-6-(naphthalen-2-ylmethylthio)pyrimidin-4-yl)phenyl)propanoic acid;
(2S)-2-Amino-3-(4-(2-amino-6-(1-(3,4-difluorophenyl)-2,2,2-trifluoroethoxy)pyrimidin-4-yl)phenyl)propanoic acid;
(2S)-2-Amino-3-(4-(2-amino-6-(2,2,2-trifluoro-1-(3′-methylbiphenyl-2-yl)ethoxy)pyrimidin-4-yl)phenyl)propanoic acid;
(S)-2-Amino-3-(4-(5-(3-(cyclopentyloxy)-4-methoxybenzylamino)pyridin-3-yl)phenyl)propanoic acid;
2-Amino-3-(3-(4-amino-6-((R)-1-(naphthalen-2-yl)ethylamino)-1,3,5-triazin-2-yl)phenyl)propanoic acid;
2-Amino-3-(4-(4-amino-6-((R)-1-(naphthalen-2-yl)ethylamino)-1,3,5-triazin-2-yl)-2-fluorophenyl)propanoic acid;
(2S)-2-Amino-3-(4-(4-amino-6-(1-(adamantyll)ethylamino)-1,3,5-triazin-2-yl)phenyl)propanoic acid;
(S)-2-Amino-3-(4-(5-fluoro-4-((R)-1-(naphthalen-2-yl)ethylamino)pyrimidin-2-yl)phenyl)propanoic acid;
(S)-2-Amino-3-(4-(2-amino-6-(4-(trifluoromethyl)-benzylamino)pyrimidin-4-yl)phenyl)propanoic acid;
2-amino-3-(5-(5-phenylthiophen-2-yl)-1H-indol-3-yl)propanoic acid;
(S)-2-amino-3-(4-(4-(4-phenoxyphenyl)-1H-1,2,3-triazol-1-yl)phenyl)propanoic acid;
(S)-2-amino-3-(4-(4-(4-(thiophene-2-carboxamido)phenyl)-1H-1,2,3-triazol-1-yl)phenyl)propanoic acid;
(S)-2-amino-3-(4-(5-(4-(thiophene-2-carboxamido)phenyl)-1H-1,2,3-triazol-1-yl)phenyl)propanoic acid;
(S)-2-amino-3-(4-(2-amino-6-(phenylethynyl)pyrimidin-4-yl)phenyl)propanoic acid;
(S)-2-Amino-3-(4-(5-(2-fluoro-4,5-dimethoxybenzylamino)pyrazin-2-yl)phenyl)propanoic acid;
(S)-2-Amino-3-(4-(2-amino-6-(4-(2-methoxyphenyl)piperidin-1-yl)pyrimidin-4-yl)phenyl)propanoic acid;
(S)-2-Amino-3-(4-(6-(3-(cyclopentyloxy)-4-methoxybenzylamino)-2-(dimethylamino)pyrimidin-4-yl)phenyl)propanoic acid;
(S)-2-Amino-3-(4-(5-(3,4-dimethylbenzylamino)pyrazin-2-yl)phenyl)propanoic acid;
(S)-2-Amino-3-(4-(5-(biphenyl-2-ylmethylamino)pyrazin-2-yl)phenyl)propanoic acid;
(S)-Ethyl 2-amino-3-(4-(2-amino-6-(4-(trifluoromethyl)benzylamino)pyrimidin-4-yl)phenyl)propanoate;
(S)-2-Amino-3-(4-(5-(cyclopentylmethylamino)pyrazin-2-yl)phenyl)propanoic acid;
(2S)-2-Amino-3-(4-(2-amino-6-(3-(2-(trifluoromethyl)phenyl)pyrrolidin-1-yl)pyrimidin-4-yl)phenyl)propanoic acid;
(2S)-2-Amino-3-(4-(2-amino-6-(1,2,3,4-tetrahydronaphthalen-1-ylamino)pyrimidin-4-yl)phenyl)propanoic acid;
(S)-2-Amino-3-(4-(2-amino-6-((R)-1-(naphthalen-2-yl)ethoxy)pyrimidin-4-yl)phenyl)propanoic acid;
(2S)-2-Amino-3-(4-(2-amino-6-(1,2-diphenylethylamino)pyrimidin-4-yl)phenyl)propanoic acid;
(S)-2-Amino-3-(4-(2-amino-6-((R)-1-(4-(benzo[b]thiophen-3-yl)phenyl)ethylamino)pyrimidin-4-yl)phenyl)propanoic acid;
(S)-2-Amino-3-(4-(4-amino-6-((R)-1-(4′-methoxybiphenyl-4-yl)ethylamino)-1,3,5-triazin-2-yl)phenyl)propanoic acid;
2-Amino-3-(1-(4-amino-6-((R)-1-(naphthalen-2-yl)ethylamino)-1,3,5-triazin-2-yl)piperidin-4-yl)propanoic acid;
(2S)-2-Amino-3-(4-(4-amino-6-(1-(4-fluoronaphthalen-1-yl)ethylamino)-1,3,5-triazin-2-yl)phenyl)propanoic acid;
(S)-2-Amino-3-(4-(4-amino-6-((3′-fluorobiphenyl-4-yl)methylamino)-1,3,5-triazin-2-yl)phenyl)propanoic acid;
2-Amino-3-(4-(4-amino-6-((R)-1-(naphthalen-2-yl)ethylamino)-1,3,5-triazin-2-yl)-2-fluorophenyl)propanoic acid;
(S)-2-Amino-3-(4-(2-amino-6-((R)-2,2,2-trifluoro-1-(3′-methoxybiphenyl-4-yl)ethoxy)pyrimidin-4-yl)phenyl)propanoic acid;
(2S)-2-Amino-3-(4-(4-amino-6-(2,2,2-trifluoro-1-(3′-fluorobiphenyl-2-yl)ethoxy)-1,3,5-triazin-2-yl)phenyl)propanoic acid;
(2S)-2-Amino-3-(4-(4-amino-6-(1-(4-tert-butylphenyl)ethylamino)-1,3,5-triazin-2-yl)phenyl)propanoic acid;
(2S)-2-Amino-3-(4-(2-amino-6-(2,2,2-trifluoro-1-(3′-fluorobiphenyl-4-yl)ethoxy)pyrimidin-4-yl)phenyl)propanoic acid;
(2S)-2-Amino-3-(4-(4-amino-6-(6,7-dihydroxy-1-methyl-3,4-dihydroisoquinolin-2(1H)-yl)-1,3,5-triazin-2-yl)phenyl)propanoic acid;
(2S)-2-Amino-3-(4-(4-amino-6-(2,2,2-trifluoro-1-(3′-methylbiphenyl-4-yl)ethoxy)-1,3,5-triazin-2-yl)phenyl)propanoic acid;
(S)-2-Amino-3-(4-(4-amino-6-((R)-1-(naphthalen-2-yl)ethylamino)pyrimidin-2-yl)phenyl)propanoic acid;
(S)-2-Amino-3-(4-(2-amino-6-(benzylthio)pyrimidin-4-yl)phenyl)propanoic acid;
(2S)-2-Amino-3-(4-(2-amino-6-(2,2,2-trifluoro-1-(4′-fluorobiphenyl-4-yl)ethoxy)pyrimidin-4-yl)phenyl)propanoic acid;
(2S)-2-Amino-3-(4-(6-(3-(4-chlorophenoxy)piperidin-1-yl)pyrimidin-4-yl)phenyl)propanoic acid;
(S)-3-(4-(4-Amino-6-((R)-1-(naphthalen-2-yl)ethylamino)-1,3,5-triazin-2-yl)phenyl)-2-(2-aminoacetamido)propanoic acid;
(S)-2-Amino-3-(4-(6-((R)-1-(naphthalen-2-yl)ethylamino)-2-(trifluoromethyl)pyrimidin-4-yl)phenyl)propanoic acid;
(S)-2-Amino-3-(4-(2-amino-6-(4-(3-chlorophenyl)piperazin-1-yl)pyrimidin-4-yl)phenyl)propanoic acid;
(S)-2-Amino-3-(4-(2-amino-6-((R)-2,2,2-trifluoro-1-phenylethoxy)pyrimidin-4-yl)phenyl)propanoic acid;
(2S)-2-Amino-3-(4-(2-amino-6-(1,4-diphenylbutylamino)pyrimidin-4-yl)phenyl)propanoic acid;
(2S)-2-Amino-3-(4-(6-(1-(3′-chlorobiphenyl-2-yl)-2,2,2-trifluoroethoxy)pyrimidin-4-yl)phenyl)propanoic acid;
(2S)-2-Amino-3-(4-(4-amino-6-(1-(biphenyl-4-yl)-2,2,2-trifluoroethoxy)-1,3,5-triazin-2-yl)phenyl)propanoic acid;
(2S)-2-Amino-3-(4-(2-amino-6-(2,2,3,3,3-pentafluoro-1-(3-fluoro-4-methylphenyl)propoxy)pyrimidin-4-yl)phenyl)propanoic acid;
(S)-Ethyl 2-amino-3-(4-(2-amino-6-((R)-2,2,2-trifluoro-1-(3′-methoxybiphenyl-4-yl)ethoxy)pyrimidin-4-yl)phenyl)propanoate;
(S)-2-Amino-3-(4-(2-amino-6-((S)-2,2,2-trifluoro-1-(3′-methoxybiphenyl-4-yl)ethoxy)pyrimidin-4-yl)phenyl)propanoic acid;
(2S)-2-Amino-3-(4-(2-amino-6-(2,2,2-trifluoro-1-(3-fluoro-3′-methoxybiphenyl-4-yl)ethoxy)pyrimidin-4-yl)phenyl)propanoic acid;
(2S)-2-Amino-3-(4-(2-amino-6-(1-(3′-(dimethylamino)biphenyl-2-yl)-2,2,2-trifluoroethoxy)pyrimidin-4-yl)phenyl)propanoic acid;
(2S)-2-Amino-3-(4-(2-amino-6-(2,2,2-trifluoro-1-(3′-methoxy-5-methylbiphenyl-2-yl)ethoxy)pyrimidin-4-yl)phenyl)propanoic acid;
(2S)-2-Amino-3-(4-(2-amino-6-(2,2,2-trifluoro-1-(4′-methoxy-5-methylbiphenyl-2-yl)ethoxy)pyrimidin-4-yl)phenyl)propanoic acid;
(2S)-2-Amino-3-(4-(2-amino-6-(2,2,2-trifluoro-1-(3′-methoxy-3-(methylsulfonyl)biphenyl-4-yl)ethoxy)pyrimidin-4-yl)phenyl)propanoic acid;
(2S)-2-Amino-3-(4-(2-amino-6-(1-(2-(cyclopropylmethoxy)-4-fluorophenyl)-2,2,2-trifluoroethoxy)pyrimidin-4-yl)phenyl)propanoic acid;
(2S)-2-Amino-3-(4-(6-(1-(2-(cyclopropylmethoxy)-4-fluorophenyl)-2,2,2-trifluoroethoxy)pyrimidin-4-yl)phenyl)propanoic acid;
(2S)-2-Amino-3-(4-(2-amino-6-(2,2,2-trifluoro-1-(2-(isopentyloxy)phenyl)ethoxy)pyrimidin-4-yl)phenyl)propanoic acid;
(2S)-2-Amino-3-(4-(5-(2,2,2-trifluoro-1-(3′-fluorobiphenyl-4-yl)ethoxy)pyrazin-2-yl)phenyl)propanoic acid;
(2S)-2-Amino-3-(4-(2-amino-6-(2,2,2-trifluoro-1-(4′-methoxybiphenyl-2-yl)ethoxy)pyrimidin-4-yl)phenyl)propanoic acid;
(2S)-2-Amino-3-(4-(2-amino-6-(1-(3′-carbamoylbiphenyl-2-yl)-2,2,2-trifluoroethoxy)pyrimidin-4-yl)phenyl)propanoic acid;
(2S)-2-Amino-3-(4-(2-amino-6-(1-(4′-carbamoylbiphenyl-2-yl)-2,2,2-trifluoroethoxy)pyrimidin-4-yl)phenyl)propanoic acid;
(2S)-2-Amino-3-(4-(2-amino-6-(2,2,2-trifluoro-1-(4-(2-methoxyphenoxy)phenyl)ethoxy)pyrimidin-4-yl)phenyl)propanoic acid;
(2S)-2-Amino-3-(4-(6-(2,2,2-trifluoro-1-(4-(2-methoxyphenoxy)phenyl)ethoxy)pyrimidin-4-yl)phenyl)propanoic acid;
(2S)-2-Amino-3-(4-(6-(2,2,2-trifluoro-1-(2-(isopentyloxy)phenyl)ethoxy)pyrimidin-4-yl)phenyl)propanoic acid;
(2S)-3-(4-(6-(1-(3′-Acetamidobiphenyl-2-yl)-2,2,2-trifluoroethoxy)-2-aminopyrimidin-4-yl)phenyl)-2-aminopropanoic acid;
(2S)-3-(4-(6-(1-(4′-Acetamidobiphenyl-2-yl)-2,2,2-trifluoroethoxy)-2-aminopyrimidin-4-yl)phenyl)-2-aminopropanoic acid;
(2S)-2-Amino-3-(4-(2-amino-6-(1-(4-cyanophenyl)-2,2,2-trifluoroethoxy)pyrimidin-4-yl)phenyl)propanoic acid;
(S)-Ethyl 2-amino-3-(4-(2-amino-6-((R)-2,2,2-trifluoro-1-p-tolylethoxy)pyrimidin-4-yl)phenyl)propanoate;
(2S)-2-Amino-3-(4-(2-amino-6-(2,2,2-trifluoro-1-(1-methoxybicyclo[2.2.2]oct-5-en-2-yl)ethoxy)pyrimidin-4-yl)phenyl)propanoic acid;
(2S)-2-Amino-3-(4-(2-amino-6-(1-(4-(cyclopentyloxy)phenyl)-2,2,2-trifluoroethoxy)pyrimidin-4-yl)phenyl)propanoic acid;
(2S)-2-Amino-3-(4-(6-(1-(4-(cyclopentyloxy)phenyl)-2,2,2-trifluoroethoxy)pyrimidin-4-yl)phenyl)propanoic acid;
(2S)-2-Amino-3-(4-(2-amino-6-(2,2,2-trifluoro-1-(4-(3-methoxyphenoxy)phenyl)ethoxy)pyrimidin-4-yl)phenyl)propanoic acid;
(2S)-2-Amino-3-(4-(2-amino-6-(1-(4,5-dimethoxybiphenyl-2-yl)-2,2,2-trifluoroethoxy)pyrimidin-4-yl)phenyl)propanoic acid;
(2S)-2-Amino-3-(4-(2-amino-6-(1-(4,5-dimethoxy-3′-methylbiphenyl-2-yl)-2,2,2-trifluoroethoxy)pyrimidin-4-yl)phenyl)propanoic acid;
(2S)-2-Amino-3-(4-(5-(2,2,2-trifluoro-1-(2′-methylbiphenyl-2-yl)ethoxy)pyrazin-2-yl)phenyl)propanoic acid;
(2S)-2-Amino-3-(4-(6-(2,2,2-trifluoro-1-(4-(3-methoxyphenoxy)phenyl)ethoxy)pyrimidin-4-yl)phenyl)propanoic acid;
(2S)-2-Amino-3-(4-(2-amino-6-(1-(2-(3,5-difluorophenoxy)phenyl)-2,2,2-trifluoroethoxy)pyrimidin-4-yl)phenyl)propanoic acid;
(2S)-2-Amino-3-(4-(2-amino-6-(2,2,2-trifluoro-1-(4-(4-methoxyphenoxy)phenyl)ethoxy)pyrimidin-4-yl)phenyl)propanoic acid;
(2S)-2-Amino-3-(4-(2-amino-6-(1-(4′-((S)-2-amino-2-carboxyethyl)biphenyl-2-yl)-2,2,2-trifluoroethoxy)pyrimidin-4-yl)phenyl)propanoic acid;
(2S)-2-Amino-3-(4-(2-amino-6-(1-(2-bromophenyl)-2,2,2-trifluoroethoxy)pyrimidin-4-yl)phenyl)propanoic acid;
(2S)-2-Amino-3-(4-(5-(2,2,2-trifluoro-1-(3′-methylbiphenyl-2-yl)ethoxy)pyrazin-2-yl)phenyl)propanoic acid;
(2S)-2-Amino-3-(4-(2-amino-6-(2,2,2-trifluoro-1-(4-methoxybiphenyl-2-yl)ethoxy)pyrimidin-4-yl)phenyl)propanoic acid;
(2S)-2-Amino-3-(4-(5-(2,2,2-trifluoro-1-(2-(4-methylthiophen-3-yl)phenyl)ethoxy)pyrazin-2-yl)phenyl)propanoic acid;
(2S)-2-Amino-3-(4-(2-amino-6-(2,2,2-trifluoro-1-(4-methoxy-3′-methylbiphenyl-2-yl)ethoxy)pyrimidin-4-yl)phenyl)propanoic acid;
(2S)-2-Amino-3-(4-(2-amino-6-(2,2,2-trifluoro-1-(3′-(hydroxymethyl)biphenyl-2-yl)ethoxy)pyrimidin-4-yl)phenyl)propanoic acid;
(2S)-2-Amino-3-(4-(2-amino-6-(1-(3′-cyanobiphenyl-2-yl)-2,2,2-trifluoroethoxy)pyrimidin-4-yl)phenyl)propanoic acid;
(2S)-2-Amino-3-(4-(6-(1-(2-(3,5-difluorophenoxy)phenyl)-2,2,2-trifluoroethoxy)pyrimidin-4-yl)phenyl)propanoic acid;
(2S)-2-Amino-3-(4-(6-(2,2,2-trifluoro-1-(4-(4-methoxyphenoxy)phenyl)ethoxy)pyrimidin-4-yl)phenyl)propanoic acid;
(2S)-2-Amino-3-(4-(2-amino-6-(2,2,2-trifluoro-1-(2-(4-methylthiazol-2-yl)thiophen-3-yl)ethoxy)pyrimidin-4-yl)phenyl)propanoic acid;
(2S)-2-Amino-3-(4-(2-amino-6-(2,2,2-trifluoro-1-(5-(4-methoxyphenyl)isoxazol-3-yl)ethoxy)pyrimidin-4-yl)phenyl)propanoic acid;
(2S)-2-Amino-3-(4-(2-amino-6-(2,2,2-trifluoro-1-(1-phenyl-5-(trifluoromethyl)-1H-pyrazol-4-yl)ethoxy)pyrimidin-4-yl)phenyl)propanoic acid;
(2S)-2-Amino-3-(4-(2-amino-6-(1-(2-(cyclohexyloxy)-4-methylphenyl)-2,2,2-trifluoroethoxy)pyrimidin-4-yl)phenyl)propanoic acid;
(2S)-2-Amino-3-(4-(2-amino-6-(1-(2-(cyclopentyloxy)-4-methylphenyl)-2,2,2-trifluoroethoxy)pyrimidin-4-yl)phenyl)propanoic acid;
(2S)-2-Amino-3-(4-(2-amino-6-(1-(benzo[d]thiazol-6-yl)-2,2,2-trifluoroethoxy)pyrimidin-4-yl)phenyl)propanoic acid;
(2S)-2-Amino-3-(4-(2-amino-6-(2,2,2-trifluoro-1-(1-methyl-1H-imidazol-5-yl)ethoxy)pyrimidin-4-yl)phenyl)propanoic acid;
(2S)-2-Amino-3-(4-(6-(1-(2-(cyclopentyloxy)-4-methylphenyl)-2,2,2-trifluoroethoxy)pyrimidin-4-yl)phenyl)propanoic acid;
(2S)-2-Amino-3-(4-(6-(1-(2-(cyclohexyloxy)-4-methylphenyl)-2,2,2-trifluoroethoxy)pyrimidin-4-yl)phenyl)propanoic acid;
(2S)-2-Amino-3-(4-(2-amino-6-(2,2,2-trifluoro-1-(pyridin-3-yl)ethoxy)pyrimidin-4-yl)phenyl)propanoic acid;
(2S)-2-Amino-3-(4-(2-amino-6-(1-(1,3-dimethyl-1H-pyrazol-5-yl)-2,2,2-trifluoroethoxy)pyrimidin-4-yl)phenyl)propanoic acid;
(S)-2-Amino-3-(4-(2-amino-6-(3-hydroxyphenyl)pyrimidin-4-yl)phenyl)propanoic acid;
(2S)-2-Amino-3-(4-(2-amino-6-(2,2,2-trifluoro-1-(3′-hydroxybiphenyl-2-yl)ethoxy)pyrimidin-4-yl)phenyl)propanoic acid;
(S)-2-Amino-3-(4-(2-amino-6-(3,5-difluorophenyl)pyrimidin-4-yl)phenyl)propanoic acid;
(2S)-2-Amino-3-(4-(2-amino-6-(1-(3′,5′-difluorobiphenyl-2-yl)-2,2,2-trifluoroethoxy)pyrimidin-4-yl)phenyl)propanoic acid;
(2S)-2-Amino-3-(4-(6-(2,2,2-trifluoro-1-(3′-fluorobiphenyl-3-yl)ethoxy)pyrazin-2-yl)phenyl)propanoic acid;
(2S)-2-Amino-3-(4-(2-amino-6-(1-(5-ethoxy-2-methyl-2,3-dihydrobenzofuran-6-yl)-2,2,2-trifluoroethoxy)pyrimidin-4-yl)phenyl)propanoic acid;
(2S)-2-Amino-3-(4-(2-amino-6-(1-(benzofuran-5-yl)-2,2,2-trifluoroethoxy)pyrimidin-4-yl)phenyl)propanoic acid;
(2S)-2-Amino-3-(4-(2-amino-6-(2,2,2-trifluoro-1-(2-m-tolylfuran-3-yl)ethoxy)pyrimidin-4-yl)phenyl)propanoic acid;
(S)-Ethyl 3-(4-(2-amino-6-((R)-2,2,2-trifluoro-1-(3′-methoxybiphenyl-4-yl)ethoxy)pyrimidin-4-yl)phenyl)-2-(2-aminoacetamido)propanoate;
(2S)-2-Amino-3-(4-(6-(2,2,2-trifluoro-1-(2-(4-methylthiophen-3-yl)phenyl)ethoxy)pyrazin-2-yl)phenyl)propanoic acid;
(2S)-2-Amino-3-(4-(2-amino-6-(2,2,2-trifluoro-1-(5-methyl-3-phenylisoxazol-4-yl)ethoxy)pyrimidin-4-yl)phenyl)propanoic acid;
(S)-2-Amino-3-(4-(2-amino-6-(3-(methylthio)phenyl)pyrimidin-4-yl)phenyl)propanoic acid;
(2S)-2-Amino-3-(4-(2-amino-6-(2,2,2-trifluoro-1-(3′-(methylthio)biphenyl-2-yl)ethoxy)pyrimidin-4-yl)phenyl)propanoic acid;
(2S)-2-Amino-3-(4-(2-amino-6-(1-(3′-((dimethylamino)methyl)biphenyl-2-yl)-2,2,2-trifluoroethoxy)pyrimidin-4-yl)phenyl)propanoic acid;
(S)-2-Amino-3-(4-(2-amino-6-(3-(trifluoromethoxy)phenyl)pyrimidin-4-yl)phenyl)propanoic acid;
(2S)-2-Amino-3-(4-(2-amino-6-(2,2,2-trifluoro-1-(3′-(trifluoromethoxy)biphenyl-2-yl)ethoxy)pyrimidin-4-yl)phenyl)propanoic acid;
(S)-3-(4-(2-Amino-6-((R)-2,2,2-trifluoro-1-(3′-methoxybiphenyl-4-yl)ethoxy)pyrimidin-4-yl)phenyl)-2-(2-aminoacetamido)propanoic acid;
(2S)-2-Amino-3-(4-(2-amino-6-(2,2,2-trifluoro-1-(1-methyl-5-phenyl-1H-pyrazol-4-yl)ethoxy)pyrimidin-4-yl)phenyl)propanoic acid;
(2S)-2-Amino-3-(4-(2-amino-6-(2,2,2-trifluoro-1-(4-(methylsulfonyl)phenyl)ethoxy)pyrimidin-4-yl)phenyl)propanoic acid;
(S)-2-Amino-3-(4-(2-amino-6-((R)-1-(3′-(dimethylamino)biphenyl-2-yl)-2,2,2-trifluoroethoxy)pyrimidin-4-yl)phenyl)propanoic acid;
(2S)-2-Amino-3-(4-(2-amino-6-(1-(2-chloro-4-(methylsulfonyl)phenyl)-2,2,2-trifluoroethoxy)pyrimidin-4-yl)phenyl)propanoic acid;
(2S)-2-Amino-3-(4-(2-amino-6-(2,2,2-trifluoro-1-(3-(furan-2-yl)thiophen-2-yl)ethoxy)pyrimidin-4-yl)phenyl)propanoic acid;
(2S)-2-Amino-3-(4-(2-amino-6-(1-(2-(cyclopentyloxy)-4-fluorophenyl)-2,2,2-trifluoroethoxy)pyrimidin-4-yl)phenyl)propanoic acid;
(2S)-2-Amino-3-(4-(2-amino-6-(2,2,2-trifluoro-1-(2-(3-methoxyphenyl)cyclohex-1-enyl)ethoxy)pyrimidin-4-yl)phenyl)propanoic acid;
(2S)-2-Amino-3-(4-(2-amino-6-(2,2,2-trifluoro-1-(pyrimidin-5-yl)ethoxy)pyrimidin-4-yl)phenyl)propanoic acid;
(2S)-2-Amino-3-(4-(5-(2,2,2-trifluoro-1-(3′-methoxybiphenyl-3-yl)ethoxy)pyrazin-2-yl)phenyl)propanoic acid;
(S)-2-Amino-3-(4-(2-amino-6-((S)-1-(3′-(dimethylamino)biphenyl-2-yl)-2,2,2-trifluoroethoxy)pyrimidin-4-yl)phenyl)propanoic acid;
(2S)-2-Amino-3-(4-(2-amino-6-(2,2,2-trifluoro-1-(2-(furan-2-carboxamido)phenyl)ethoxy)pyrimidin-4-yl)phenyl)propanoic acid;
(2S)-2-Amino-3-(4-(2-amino-6-(1-(4-chloro-2-(methylsulfonyl)phenyl)-2,2,2-trifluoroethoxy)pyrimidin-4-yl)phenyl)propanoic acid;
(S)-Isopropyl 2-amino-3-(4-(2-amino-6-((R)-2,2,2-trifluoro-1-(3′-methoxybiphenyl-4-yl)ethoxy)pyrimidin-4-yl)phenyl)propanoate;
(2S)-2-Amino-3-(4-(6-(1-(2-(cyclopentyloxy)-4-fluorophenyl)-2,2,2-trifluoroethoxy)pyrimidin-4-yl)phenyl)propanoic acid;
(2S)-2-Amino-3-(4-(6-(1-(2-(cyclohexyloxy)-4-fluorophenyl)-2,2,2-trifluoroethoxy)pyrimidin-4-yl)phenyl)propanoic acid;
(2S)-2-Amino-3-(4-(2-amino-6-(2,2,2-trifluoro-1-(1-(thiophen-2-yl)cyclohexyl)ethoxy)pyrimidin-4-yl)phenyl)propanoic acid;
(2S)-2-Amino-3-(4-(2-(2,2,2-trifluoro-1-(3′-methoxybiphenyl-4-yl)ethoxy)thiazol-5-yl)phenyl)propanoic acid;
(2S)-2-Amino-3-(4-(2-amino-6-(1-(2-(cyclohexyloxy)-4-fluorophenyl)-2,2,2-trifluoroethoxy)pyrimidin-4-yl)phenyl)propanoic acid;
(2S)-2-Amino-3-(4-(2-amino-6-(2,2,2-trifluoro-1-(1-(4-methoxyphenyl)cyclohexyl)ethoxy)pyrimidin-4-yl)phenyl)propanoic acid;
(2S)-2-Amino-3-(4-(6-(2,2,2-trifluoro-1-(4-fluoro-2-methylphenyl)ethoxy)pyrimidin-4-yl)phenyl)propanoic acid;
(2S)-2-Amino-3-(4-(2-amino-6-(2,2,2-trifluoro-1-(4-fluoro-2-methylphenyl)ethoxy)pyrimidin-4-yl)phenyl)propanoic acid;
(2S)-2-Amino-3-(4-(2-amino-6-(oxazol-2-yl(phenyl)methoxy)pyrimidin-4-yl)phenyl)propanoic acid;
(S)-2-Amino-3-(4-(2-amino-6-(1-cyclohexyl-2,2,2-trifluoroethylideneaminooxy)pyrimidin-4-yl)phenyl)propanoic acid;
(2S)-2-Amino-3-(4-(2-amino-6-(1-(2-(3-(dimethylamino)phenyl)furan-3-yl)-2,2,2-trifluoroethoxy)pyrimidin-4-yl)phenyl)propanoic acid;
(2S)-2-Amino-3-(4-(2-amino-6-(2,2,2-trifluoro-1-(5-phenylthiophen-2-yl)ethoxy)pyrimidin-4-yl)phenyl)propanoic acid;
(S)-Phenyl 2-amino-3-(4-(2-amino-6-((R)-2,2,2-trifluoro-1-(3′-methoxybiphenyl-4-yl)ethoxy)pyrimidin-4-yl)phenyl)propanoate;
(S)-2-Amino-3-(4-(2-amino-6-((R)-1-(3′-((dimethylamino)methyl)biphenyl-4-yl)-2,2,2-trifluoroethoxy)pyrimidin-4-yl)phenyl)propanoic acid;
(S)-2-Amino-3-(4-(1-(3-methoxybenzoyl)-1H-pyrazol-4-yl)phenyl)propanoic acid;
(2S)-2-Amino-3-(4-(6-(2,2,2-trifluoro-1-(5-phenylfuran-2-yl)ethoxy)pyrimidin-4-yl)phenyl)propanoic acid;
(2S)-2-Amino-3-(4-(2-amino-6-(1-(4-chloro-2-fluorophenyl)-2,2,2-trifluoroethoxy)pyrimidin-4-yl)phenyl)propanoic acid;
(S,E)-2-Amino-3-(4-(2-amino-6-(4-(trifluoromethyl)styryl)pyrimidin-4-yl)phenyl)propanoic acid;
(2S)-2-Amino-3-(4-(2-amino-6-(1-(3,4-dichlorophenyl)-2,2,2-trifluoroethoxy)pyrimidin-4-yl)phenyl)propanoic acid;
(2S)-2-Amino-3-(4-(2-amino-6-(1-(4-chloro-3-fluorophenyl)-2,2,2-trifluoroethoxy)pyrimidin-4-yl)phenyl)propanoic acid;
(S)-2-Amino-3-(4-(2-amino-6-((R)-1-(3′-(dimethylamino)biphenyl-4-yl)-2,2,2-trifluoroethoxy)pyrimidin-4-yl)phenyl)propanoic acid;
(2S)-2-Amino-3-(4-(2-amino-6-(1-chloro-2,2,2-trifluoro-1-(4-methoxybiphenyl-2-yl)ethoxy)pyrimidin-4-yl)phenyl)propanoic acid;
(2S)-2-Amino-3-(4-(6-(2,2,2-trifluoro-1-(5-phenylthiophen-2-yl)ethoxy)pyrimidin-4-yl)phenyl)propanoic acid;
(S)-2-Amino-3-(4-(5-(4-phenoxyphenyl)-1H-1,2,3-triazol-1-yl)phenyl)propanoic acid;
(S,E)-2-Amino-3-(4-(2-amino-6-(2-(biphenyl-4-yl)vinyl)pyrimidin-4-yl)phenyl)propanoic acid;
(S)-2-Amino-3-(4-(4-amino-6-((R)-2,2,2-trifluoro-1-(3′-methoxybiphenyl-4-yl)ethoxy)pyrimidin-2-yl)phenyl)propanoic acid;
(S)-2-Amino-3-(4-(4′-methoxybiphenyl-4-ylsulfonamido)phenyl)propanoic acid;
(2S)-2-Amino-3-(4-(2-amino-6-(2,2,2-trifluoro-1-(6-(3-methoxyphenyl)pyridin-3-yl)ethoxy)pyrimidin-4-yl)phenyl)propanoic acid;
(2S)-2-Amino-3-(4-(2-amino-6-(2,2,2-trifluoro-1-(6-(2-fluoro-3-methoxyphenyl)pyridin-3-yl)ethoxy)pyrimidin-4-yl)phenyl)propanoic acid;
2-Amino-3-(5-(4′-methylbiphenyl-4-yl)-1H-indol-3-yl)propanoic acid;
2-Amino-3-(5-m-tolyl-1H-indol-3-yl)propanoic acid;
(2S)-2-Amino-3-(4-(2-(2-methoxyphenyl)furan-3-carboxamido)phenyl)propanoic acid;
2-Amino-3-(5-(1-benzyl-1H-pyrazol-4-yl)-1H-indol-3-yl)propanoic acid;
(2S)-2-Amino-3-(4-(2-amino-6-(2,2,2-trifluoro-1-(6-(thiophen-2-yl)pyridin-3-yl)ethoxy)pyrimidin-4-yl)phenyl)propanoic acid;
2-Amino-3-(6-(1-benzyl-1H-pyrazol-4-yl)-1H-indol-3-yl)propanoic acid;
(S)-2-Amino-3-(4-((2-(4-(trifluoromethyl)phenyl)thiazol-4-yl)methylamino)phenyl)propanoic acid;
(S)-2-Amino-3-(4-((4′-methoxybiphenyl-4-ylsulfonamido)methyl)phenyl)propanoic acid;
(S)-2-Amino-3-(4-(3-(2-methoxydibenzo[b,d]furan-3-yl)ureido)phenyl)propanoic acid;
(S)-2-Amino-3-(4-(3-(2,2-diphenylethyl)ureido)phenyl)propanoic acid;
(S)-2-Amino-3-(4-(phenylethynyl)phenyl)propanoic acid;
(S)-2-Amino-3-(4-(2-amino-6-((5-(1-methyl-5-(trifluoromethyl)-1H-pyrazol-3-yl)thiophen-2-yl)methoxy)pyrimidin-4-yl)phenyl)propanoic acid;
(2S)-2-Amino-3-(4-(2-amino-6-(1,1,1-trifluoro-3-((R)-2,2,3-trimethylcyclopent-3-enyl)propan-2-yloxy)pyrimidin-4-yl)phenyl)propanoic acid;
(2S)-2-Amino-3-(4-(2-amino-6-(3-(2-hydroxyethylcarbamoyl)piperidin-1-yl)pyrimidin-4-yl)phenyl)propanoic acid;
(2S)-2-Amino-3-(4-(2-amino-6-(3-(pyridin-2-yloxy)piperidin-1-yl)pyrimidin-4-yl)phenyl)propanoic acid;
(S)-2-Amino-3-(4-(2-amino-6-(4-chloro-3-(piperidine-1-carbonyl)phenyl)pyrimidin-4-yl)phenyl)propanoic acid;
(S)-2-Amino-3-(4-{2-amino-6-[2,2,2-trifluoro-1-(4-pyridin-4-yl-phenyl)-ethoxy]-pyrimidin-4-yl}-phenyl)-propionic acid;
(S)-2-Amino-3-(4-{6-[2,2,2-trifluoro-1-(2-pyridin-4-yl-phenyl)-ethoxy]-pyrimidin-4-yl}-phenyl)-propionic acid;
(S)-2-Amino-3-(4-{2-amino-6-[2,2,2-trifluoro-1-(2-(4-methyl-thiophen-3-yl)-phenyl]-ethoxy}-pyrimidin-4-yl)-phenyl]-propionic acid;
(S)-2-Amino-3-(4-{2-amino-6-[2,2,2-trifluoro-1-(2-(5-methyl-thiophen-3-yl)-phenyl]-ethoxy}-pyrimidin-4-yl)-phenyl]-propionic acid;
(S)-2-Amino-3-(4-{2-amino-6-[2,2,2-trifluoro-1-(4-furan-3-yl-phenyl)-ethoxy]-pyrimidin-4-yl}-phenyl)-propionic acid;
(S)-2-Amino-3-[4-{2-amino-6-{1-[2-(5-dimethylaminomethyl-furan-2-yl)-phenyl]-2,2,2-trifluoro-ethoxy}-pyrimidin-4-yl)-phenyl]-propionic acid;
(S)-2-Amino-3-[4-(2-amino-6-{1-[2-(6-cyano-pyridin-3-yl)-phenyl]-2,2,2-trifluoro-ethoxy}-pyrimidin-4-yl)-phenyl]-propionic acid;
(S)-2-Amino-3-(4-{2-amino-6-[2,2,2-trifluoro-1-(2-imidazol-1-yl-phenyl)-ethoxy]-pyrimidin-4-yl}-phenyl)-propionic acid;
(S)-2-Amino-3-(4-{6-[2,2,2-trifluoro-1-(2-pyrazol-1-yl-phenyl)-ethoxy]-pyrimidin-4-yl}-phenyl)-propionic acid;
(S)-2-amino-3-[4-(2-amino-6-{2,2,2-trifluoro-1-[2-(3-trifluoromethyl-pyrazol-1-yl)-phenyl]-ethoxy}-pyrimidin-4-yl)-phenyl]-propionic acid;
(S)-2-Amino-3-[4-(2-amino-6-{1-[2-(3,5-dimethyl-pyrazol-1-yl)-phenyl]-2,2,2-trifluoro-ethoxy}-pyrimidin-4-yl)-phenyl]-propionic acid;
(S)-2-Amino-3-[4-(2-amino-6-{2,2,2-trifluoro-1-[2-(3-phenyl-pyrazol-1-yl)-phenyl]-ethoxy}-pyrimidin-4-yl)-phenyl]-propionic acid;
(S)-2-Amino-3-[4-(2-amino-6-{2,2,2-trifluoro-1-[5-methoxy-2-(4-methyl-pyrazol-1-yl)-phenyl]-ethoxy}-pyrimidin-4-yl)-phenyl]-propionic acid;
(S)-2-amino-3-[4-(2-amino-6-{(R)-2,2,2-trifluoro-1-[2-(3-methyl-pyrazol-1-yl)-phenyl]-ethoxy}-pyrimidin-4-yl)-phenyl]-propionic acid;
(S)-2-amino-3-[4-(2-amino-6-{1-[4-chloro-2-(3-methyl-pyrazol-1-yl)-phenyl]-2,2,2-trifluoro-ethoxy}-pyrimidin-4-yl)-phenyl]-propionic acid;
(S)-2-Amino-3-[4-(2-amino-6-{R-1-[4-chloro-2-(3-methyl-pyrazol-1-yl)-phenyl]-2,2,2-trifluoro-ethoxy}-pyrimidin-4-yl)-phenyl]-propionic acid ethyl ester;
(S)-2-amino-3-(4-(2-amino-6-((R)-1-(4-chloro-2-(3-methyl-1H-pyrazol-1-yl)phenyl)-2,2,2-trifluoroethoxy)pyrimidin-4-yl)phenyl)propanoic acid;
(S)-2-Amino-3-(4-{2-amino-6-[2,2,2-trifluoro-1-(2-thiazol-2-yl-phenyl)-ethoxy]-pyrimidin-4-yl}-phenyl)-propionic acid;
(S)-2-Amino-3-[4-(2-amino-6-{2,2,2-trifluoro-1-[2-(pyridin-3-yloxy)-phenyl]-ethoxy}-pyrimidin-4-yl)-phenyl]-propionic acid;
(S)-2-Amino-3-[4-(2-amino-6-{2,2,2-trifluoro-1-[4-(pyridin-3-yloxy)-phenyl]-ethoxy}-pyrimidin-4-yl)-phenyl]-propionic acid;
(S)-2-Amino-3-[4-(6-{2,2,2-trifluoro-1-[4-(pyridin-3-yloxy)-phenyl]-ethoxy}-pyrimidin-4-yl)-phenyl]-propionic acid;
(S)-2-Amino-3-(4-{2-amino-6-[2,2,2-trifluoro-1-(4-thiophen-2-yl-phenyl)-ethoxy]-pyrimidin-4-yl}-phenyl)-propionic acid;
(S)-2-Amino-3-(4-{6-[2,2,2-trifluoro-1-(4-imidazol-1-yl-phenyl)-ethoxy]-pyrimidin-4-yl}-phenyl)-propionic acid;
(S)-2-Amino-3-(4-{2-amino-6-[2,2,2-trifluoro-1-(4-[1,2,4]triazol-1-yl-phenyl)-ethoxy]-pyrimidin-4-yl}-phenyl)-propionic acid;
(S)-2-Amino-3-(4-{2-amino-6-[2,2,2-trifluoro-1-(4-fluoro-2-thiophen-3-yl-phenyl)ethoxy]-pyrimidin-4-yl}-phenyl)-propionic acid;
(S)-2-Amino-3-[4-(2-amino-6-{2,2,2-trifluoro-1-[4-fluoro-2-(4-methyl-thiophen-2-yl)-phenyl]-ethoxy}-pyrimidin-4-yl)-phenyl]-propionic acid;
(S)-2-Amino-3-[4-(2-amino-6-{1-[2-(3,5-dimethyl-isoxazol-4-yl)-4-fluoro-phenyl]-2,2,2-trifluoro-ethoxy}-pyrimidin-4-yl)-phenyl]-propionic acid;
(S)-2-amino-3-[4-(2-amino-6-{2,2,2-trifluoro-1-[5-fluoro-2-(3-methyl-pyrazol-1-yl)-phenyl]-ethoxy}-pyrimidin-4-yl)-phenyl]-propionic acid;
(S)-2-amino-3-[4-(2-amino-6{2,2,2-trifluoro-1-[5-chloro-2-(3-methyl-pyrazol-1-yl)-phenyl]-ethoxy}-pyrimidin-4-yl)-phenyl]-propionic acid;
(S)-2-amino-3-[4-(2-amino-6-{2,2,2-trifluoro-1-[4-(2-oxo-pyrrolidin-1-yl)-phenyl]-ethoxy}-pyrimidin-4-yl)-phenyl]-propionic acid;
(S)-2-Amino-3-[4-(2-amino-6-{(R)-2,2,2-trifluoro-1-[5-fluoro-2-(3-methyl-pyrazol-1-yl)-phenyl]-ethoxy}-pyrimidin-4-yl)-phenyl]-propionic acid;
(S)-2-Amino-3-[4-(2-amino-6-{2,2,2-trifluoro-1-[4-(6-methoxy-pyridin-2-yl)-phenyl]-ethoxy}-pyrimidin-4-yl)-phenyl]-propionic acid;
(S)-2-Amino-3-[4-(2-amino-6-{2,2,2-trifluoro-1-[2-fluoro-4-(5-methoxy-pyridin-3-yl)-phenyl]-ethoxy}-pyrimidin-4-yl)-phenyl]-propionic acid;
(S)-2-Amino-3-[4-(2-amino-6-{(S)-2,2,2-trifluoro-1-[4-(2-fluoro-pyridin-4-yl)-phenyl]-ethoxy}-pyrimidin-4-yl)-phenyl]-propionic acid;
(S)-2-Amino-3-[4-(2-amino-6-{(S)-2,2,2-trifluoro-1-[4-(5-methoxy-pyridin-3-yl)-phenyl]-ethoxy}-pyrimidin-4-yl)-phenyl]-propionic acid;
(S)-2-Amino-3-[4-(2-amino-6-{(S)-2,2,2-trifluoro-1-[4-(4-trifluoromethyl-pyridin-3-yl)-phenyl]-ethoxy}-pyrimidin-4-yl)-phenyl]-propionic acid;
(S)-2-Amino-3-(4-{2-amino-6-[(S)-2,2,2-trifluoro-1-(4-isoxazol-4-yl-phenyl)-ethoxy]-pyrimidin-4-yl}-phenyl)-propionic acid;
(S)-2-Amino-3-(4-{2-amino-6-[2,2,2-trifluoro-1-(2-pyrimidin-5-yl-phenyl)-ethoxy]-pyrimidin-4-yl}-phenyl)-propionic acid;
(S)-2-amino-3-(4-{2-amino-6-[2,2,2-trifluoro-1-(2-thiophen-3-yl-phenyl)-ethoxy]-pyrimidin-4-yl}-phenyl)-propionic acid;
(S)-2-Amino-3-[4-(2-amino-6-{2,2,2-trifluoro-1-[2-(1-methyl-1H-pyrazol-4-yl)-phenyl]-ethoxy}-pyrimidin-4-yl)-phenyl]-propionic acid;
(S)-2-amino-3-(4-{6-[2,2,2-trifluoro-1-(2-furan-3-yl-phenyl)-ethoxy]-pyrimidin-4-yl}-phenyl)-propionic acid;
(S)-2-amino-3-(4-{6-[2,2,2-trifluoro-1-(2-furan-2-yl-phenyl)-ethoxy]-pyrimidin-4-yl}-phenyl)-propionic acid;
(2S)-2-amino-3-(4-(2-amino-6-(2,2,2-trifluoro-1-(4-(pyridin-3-yl)phenyl)ethoxy)pyrimidin-4-yl)phenyl)propanoic acid;
(2S)-2-amino-3-(4-(2-amino-6-(2,2,2-trifluoro-1-(2-(2-methylpyridin-4-yl)phenyl)ethoxy)pyrimidin-4-yl)phenyl)propanoic acid;
(2S)-2-amino-3-(4-(2-amino-6-(2,2,2-trifluoro-1-(2-(4-methylthiophen-3-yl)phenyl)ethoxy)pyrimidin-4-yl)phenyl)propanoic acid;
(2S)-3-(4-(6-(1-(2-(1H-pyrazol-1-yl)phenyl)-2,2,2-trifluoroethoxy)-2-aminopyrimidin-4-yl)phenyl)-2-aminopropanoic acid;
(2S)-2-amino-3-(4-(2-amino-6-(2,2,2-trifluoro-1-(4-(furan-2-yl)phenyl)ethoxy)pyrimidin-4-yl)phenyl)propanoic acid;
(2S)-2-amino-3-(4-(6-(2,2,2-trifluoro-1-(2-(pyridin-3-yloxy)phenyl)ethoxy)pyrimidin-4-yl)phenyl)propanoic acid;
(2S)-3-(4-(6-(1-(2-(1H-1,2,4-triazol-1-yl)phenyl)-2,2,2-trifluoroethoxy)-2-aminopyrimidin-4-yl)phenyl)-2-aminopropanoic acid;
(2S)-2-amino-3-(4-(2-amino-6-(2,2,2-trifluoro-1-(2-(furan-3-yl)phenyl)ethoxy)pyrimidin-4-yl)phenyl)propanoic acid;
(2S)-2-amino-3-(4-(2-amino-6-(2,2,2-trifluoro-1-(4-(furan-2-yl)-3-methoxyphenyl)ethoxy)pyrimidin-4-yl)phenyl)propanoic acid;
(2S)-2-amino-3-(4-(5-(2,2,2-trifluoro-1-(2-(furan-2-yl)phenyl)ethoxy)pyrazin-2-yl)phenyl)propanoic acid;
(2S)-3-(4-(5-(1-(2-(1H-pyrazol-1-yl)phenyl)-2,2,2-trifluoroethoxy)pyrazin-2-yl)phenyl)-2-aminopropanoic acid;
(2S)-2-amino-3-(4-(2-amino-6-(1-(4,5-dimethoxy-2-(1H-pyrazol-1-yl)phenyl)-2,2,2-trifluoroethoxy)pyrimidin-4-yl)phenyl)propanoic acid;
(2S)-2-amino-3-(4-(2-amino-6-(2,2,2-trifluoro-1-(2-(2-methyl-1H-imidazol-1-yl)phenyl)ethoxy)pyrimidin-4-yl)phenyl)propanoic acid;
(2S)-2-amino-3-(4-(2-amino-6-(2,2,2-trifluoro-1-(2-(5-methylthiophen-2-yl)phenyl)ethoxy)pyrimidin-4-yl)phenyl)propanoic acid;
(2S)-2-amino-3-(4-(2-amino-6-(1-(2-(5-(dimethylcarbamoyl)furan-2-yl)phenyl)-2,2,2-trifluoroethoxy)pyrimidin-4-yl)phenyl)propanoic acid;
(2S)-2-amino-3-(4-(2-amino-6-(2,2,2-trifluoro-1-(4-fluoro-2-(thiophen-2-yl)phenyl)ethoxy)pyrimidin-4-yl)phenyl)propanoic acid;
(2S)-2-amino-3-(4-(6-(2,2,2-trifluoro-1-(4-fluoro-2-(thiophen-2-yl)phenyl)ethoxy)pyrimidin-4-yl)phenyl)propanoic acid;
(2S)-2-amino-3-(4-(6-(2,2,2-trifluoro-1-(4-fluoro-2-(thiophen-3-yl)phenyl)ethoxy)pyrimidin-4-yl)phenyl)propanoic acid;
(2S)-2-amino-3-(4-(6-(2,2,2-trifluoro-1-(4-fluoro-2-(4-methylthiophen-2-yl)phenyl)ethoxy)pyrimidin-4-yl)phenyl)propanoic acid;
(S)-2-amino-3-(4-(2-amino-6-((R)-2,2,2-trifluoro-1-(4-(6-fluoropyridin-3-yl)phenyl)ethoxy)pyrimidin-4-yl)phenyl)propanoic acid;
(2S)-3-(4-(6-(1-(4-(1H-imidazol-1-yl)phenyl)-2,2,2-trifluoroethoxy)-2-aminopyrimidin-4-yl)phenyl)-2-aminopropanoic acid;
(2S)-2-amino-3-(4-(6-(2,2,2-trifluoro-1-(4-(thiophen-2-yl)phenyl)ethoxy)pyrimidin-4-yl)phenyl)propanoic acid;
(S)-2-amino-3-(4-(2-amino-6-((R)-2,2,2-trifluoro-1-(4-(pyrimidin-5-yl)phenyl)ethoxy)pyrimidin-4-yl)phenyl)propanoic acid;
(2S)-2-amino-3-(4-(6-(1-(2-(3,5-dimethylisoxazol-4-yl)-4-fluorophenyl)-2,2,2-trifluoroethoxy)pyrimidin-4-yl)phenyl)propanoic acid;
(S)-2-amino-3-(4-(2-amino-6-((R)-2,2,2-trifluoro-1-(4-(2-methylpyridin-4-yl)phenyl)ethoxy)pyrimidin-4-yl)phenyl)propanoic acid;
(2S)-3-(4-(6-(1-(4-(1H-1,2,4-triazol-1-yl)phenyl)-2,2,2-trifluoroethoxy)pyrimidin-4-yl)phenyl)-2-aminopropanoic acid;
(2S)-2-amino-3-(4-(2-amino-6-(2,2,2-trifluoro-1-(4-(piperidin-1-ylmethyl)phenyl)ethoxy)pyrimidin-4-yl)phenyl)propanoic acid;
(2S)-2-amino-3-(4-(2-amino-6-(2,2,2-trifluoro-1-(2-fluoro-4-(2-methylpyridin-4-yl)phenyl)ethoxy)pyrimidin-4-yl)phenyl)propanoic acid;
(2S)-2-amino-3-(4-(2-amino-6-(1-(4-(6-chloropyridazin-3-yl)phenyl)-2,2,2-trifluoroethoxy)pyrimidin-4-yl)phenyl)propanoic acid;
(2S)-2-amino-3-(4-(2-amino-6-(1-(4-(4-tert-butylthiazol-2-yl)phenyl)-2,2,2-trifluoroethoxy)pyrimidin-4-yl)phenyl)propanoic acid;
(2S)-2-amino-3-(4-(2-amino-6-(2,2,2-trifluoro-1-(3′-methoxy-3-(3-methyl-1H-pyrazol-1-yl)biphenyl-4-yl)ethoxy)pyrimidin-4-yl)phenyl)propanoic acid;
(S)-ethyl-2-amino-3-(4-(2-amino-6-((R)-1-(4-chloro-2-(3-methyl-1H-pyrazol-1-yl)phenyl)-2,2,2-trifluoroethoxy)pyrimidin-4-yl)phenyl)propanoate;
(2S)-2-amino-3-(4-(2-amino-6-(1-(5-chloro-2-(3-methyl-1H-pyrazol-1-yl)phenyl)-2,2,2-trifluoroethoxy)pyrimidin-4-yl)phenyl)propanoic acid;

and pharmaceutically acceptable salts thereof.

TPH inhibitors are preferably administered in pharmaceutical compositions suitable for administration to patients. Pharmaceutical compositions include those suitable for oral, mucosal (e.g., nasal, sublingual, vaginal, buccal, or rectal), parenteral (e.g., subcutaneous, intravenous, bolus injection, intramuscular, or intraarterial), transdermal, topical and ophthalmic (e.g., topical, intravitreal) administration.

Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

### 5.3. METHODS OF TREATMENT, ASSAYS, AND KITS

This invention encompasses methods of treating IBS patients, particularly non-constipating IBS patients, who are more likely to respond to TPH inhibitor therapy than not. The methods are based, in part, on Applicants' discovery of a correlation between the effect on 5-HT and 5-HIAA levels in response to TPH inhibition in non-constipating IBS patients and the reduction in those patients' symptoms (e.g., global relief of symptoms, stool consistency).

Methods of assaying for (e.g., determining levels of) 5-HT levels in blood—which includes whole blood, blood plasma, and blood serum—are well known in the art. See, e.g., Houghton, L. A., et al., Gut 52:663-670 (2003); Kilkens, T. O. C., et al., Gut 53:1794-1800 (2004); Atkinson, W. et al., Gastroenterology 130:34-43 (2006); Liu, Q. et al., JPET 325:47-55 (2008). Methods of assaying for 5-HIAA in urine and blood are also well known in the art. Id.; Miller, A. G., et al., J. Chromatography B 878:695-699 (2010).

One embodiment of the invention encompasses a method of treating or managing non-constipating IBS, which comprises administering to a patient suffering from non-constipating IBS a therapeutically or prophylactically effective amount of a TPH inhibitor, wherein the patient suffering from non-constipating IBS exhibits a baseline peripheral 5-HT level that is greater than the average peripheral 5-HT level exhibited by people who do not suffer from IBS. In particular methods, peripheral 5-HT levels are determined by measuring 5-HT in blood plasma. In some, the measurement of 5-HT is made when the subjects are fasted (e.g., before a meal). In others, the measurement of 5-HT is made at least about 0.5, 1.0, 2.0, 3.0, or 4.0 hours after a meal. In this context, the term “about” means±0.1 hours. In some, the measurement of 5-HT is an average of a plurality of measurements taken over a period of time (e.g., 24, 48, 72 hours). In some, the baseline peripheral 5-HT level of the patient is at least about 10, 15, 20, 25, 30, 35, or 40 percent greater than the average peripheral 5-HT level exhibited by a population that does not suffer from IBS (e.g., non-constipating IBS). In this context, the term “about” means±2 percent. Average peripheral 5-HT levels exhibited by a population that does not suffer from IBS can be readily determined by simply assaying for 5-HT in such people, and averaging the results.

Another embodiment encompasses a method of treating or managing non-constipating IBS, which comprises administering to a patient suffering from non-constipating IBS a therapeutically or prophylactically effective amount of a TPH inhibitor, wherein the patient suffering from non-constipating IBS exhibits a baseline 5-HIAA level that is greater than the average 5-HIAA level exhibited by a population that does not suffer from IBS (e.g., non-constipating IBS). In some methods, the 5-HIAA is blood (e.g., plasma) 5-HIAA. In some, it is urinary 5-HIAA. In some, the measurement of 5-HIAA is obtained from urine collected over at least about 12, 24, or 48 hours. In some, the baseline 5-HIAA level of the patient is at least about 10, 15, 20, 25, 30, 35, or 40 percent greater than the average 5-HIAA level exhibited by a population that does not suffer from IBS. In this context, the term “about” means±2 percent. Average 5-HIAA levels exhibited by people who do not suffer from IBS can be readily determined by simply assaying for 5-HIAA in such people, and averaging the results.

Another embodiment encompasses a method of treating or managing non-constipating IBS, which comprises administering to a patient suffering from IBS a therapeutically or prophylactically effective amount of a TPH inhibitor, wherein the patient exhibits a whole blood 5-HT level that is greater than about 140, 145, 150, 155, or 160 ng/mL. In this context, the term “about” means±5 ng/mL.

In some methods, the blood 5-HT level is measured when the patient is fasted (e.g., before a meal). In others, the blood 5-HT level is measured at least about 2.0, 3.0, or 4.0 hours after a meal. In this context, the term “about” means±0.1 hours.

Another embodiment encompasses a method of treating or managing non-constipating IBS, which comprises administering to a patient suffering from non-constipating IBS a therapeutically or prophylactically effective amount of a TPH inhibitor, wherein the patient exhibits a baseline urinary 5-HIAA level of greater than about 3.0, 3.5, or 3.75 mg/day. In this context, the term “about” means±0.1 mg/day.

Another embodiment encompasses a method of treating or managing non-constipating IBS, which comprises administering to a patient suffering from non-constipating IBS an amount of a TPH inhibitor sufficient to lower the patient's blood 5-HT level by at least about 10, 20, 30 or 40 ng/mL compared to baseline (e.g., the level measured within 1, 2, or 3 weeks prior to the start of treatment). In this context, the term “about” means±2 ng/mL. In some methods, the blood 5-HT level is determined by measuring 5-HT in blood plasma. In some, the 5-HT level is measured when the patient is fasted (e.g., before a meal). In others, the 5-HT level is measured at least about 2.0, 3.0, or 4.0 hours after a meal. In this context, the term “about” means±0.1 hours.

Another embodiment encompasses a method of treating or managing non-constipating IBS, which comprises administering to a patient suffering from non-constipating IBS an amount of a TPH inhibitor sufficient to lower the patient's 5-HIAA level by at least about 10, 20, 30, or 40 percent compared to baseline (e.g., the level measured within 1, 2 or 3 weeks prior to the start of treatment). In this context, the term “about” means±2 percent. In some methods, the 5-HIAA level is determined by measuring blood (e.g., plasma) 5-HIAA. In others, it is determined by measuring urinary 5-HIAA. In some, the measurement of 5-HIAA is obtained from urine collected over at least 12, 24, or 48 hours.

In some methods of the invention, the TPH inhibitor is administered orally. In some, the TPH inhibitor is administered at least once daily.

This invention also encompasses methods and kits for determining whether a non-constipating IBS patient is likely to respond to TPH inhibitor therapy. For example, one embodiment of the invention encompasses a method of determining if a patient suffering from IBS will be responsive to TPH inhibitor therapy, which comprises determining if a single dose of a TPH inhibitor is sufficient to lower the patient's blood 5-HT level by at least 10, 15, or 20 ng/mL compared to baseline. In this context, the term “about” means±2 ng/mL. In a particular method, the blood is whole blood. In one method, the 5-HT level is a fasting 5-HT level.

A particular method comprises determining a patient's blood (e.g., plasma) 5-HT level, administering a TPH inhibitor to the patient, and then again determining the patient's blood 5-HT level within about 0.5, 1, 2, or 3 hours of dosing. In this context, the term “about” means±0.1 hours.

Another embodiment encompasses a method of determining if a patient suffering from non-constipating IBS will be responsive to TPH inhibitor therapy, which comprises determining if a single dose of a TPH inhibitor is sufficient to lower the patient's 5-HIAA level by at least about 20, 30, or 40 percent compared to baseline. In this context, the term “about” means±2 percent. In one method, 5-HIAA is urinary 5-HIAA (e.g., collected over a 24 hour time period). In another, 5-HIAA is blood (e.g., plasma) 5-HIAA. In one method, the 5-HIAA level is a fasting 5-HIAA level.

A particular method comprises determining a patient's blood (e.g., plasma) 5-HIAA level, administering a TPH inhibitor to the patient, and then again determining the patient's blood 5-HIAA level within about 0.5, 1, 2, or 3 hours of dosing. In this context, the term “about” means±0.1 hours.

Another embodiment encompasses a method of determining if a patient suffering from IBS will be responsive to TPH inhibitor therapy, which comprises determining: a) if the patient experiences abdominal pain or discomfort at least 3 days/month, which is associated with two or more of: improvement with defecation, onset associated with a change in frequency of stool, or onset associated with a change in form (appearance) of stool; and b) if the patient's whole blood 5-HT level is greater than about 140, 145, 150, 155, or 160 ng/mL. In this context, the term “about” means±5 ng/mL.

Another embodiment encompasses a method of determining if a patient suffering from IBS will be responsive to TPH inhibitor therapy, which comprises determining: a) if the patient experiences abdominal pain or discomfort at least 3 days/month, which is associated with two or more of: improvement with defecation, onset associated with a change in frequency of stool, or onset associated with a change in form (appearance) of stool; and b) if the patient's urinary 5-HIAA level is greater than about 3.0, 3.5, or 3.75 mg/day. In this context, the term “about” means±0.1 mg/day.

Another embodiment encompasses a method of estimating the likelihood that a patient suffering from non-constipating IBS will be responsive to TPH inhibitor therapy, which comprises: measuring the amount of 5-HT in the blood of the patient; and correlating that amount with a TPH inhibitor responder rate, wherein the TPH inhibitor responder rate is a predicted likelihood of relief from IBS symptoms for patients having a baseline 5-HT level that falls within a predetermined range. In some methods, the TPH inhibitor responder rate for the predetermined range is the percent of IBS patients (e.g., patients in a blinded clinical study of the TPH inhibitor) who previously reported experiencing relief from IBS symptoms and who had baseline 5-HT levels that fell within the predetermined range. In some methods, the TPH inhibitor responder rate for the predetermined range is the percent of IBS patients (e.g., patients in a blinded clinical study of the TPH inhibitor) who previously reported a decrease in mean stool consistency (e.g., as measured on the Bristol Stool Scale) and who had baseline 5-HT levels that fell within the predetermined range.

Another embodiment encompasses a method of estimating the likelihood that a patient suffering from IBS will be responsive to TPH inhibitor therapy, which comprises: measuring the amount of 5-HIAA in the blood or urine of the patient; and correlating that amount with a TPH inhibitor responder rate, wherein the TPH inhibitor responder rate is a predicted likelihood of relief from IBS symptoms for patients having a baseline 5-HIAA level that falls within a predetermined range. In some methods, the TPH inhibitor responder rate for the predetermined range is the percent of IBS patients (e.g., patients in a blinded clinical study of the TPH inhibitor) who previously reported experiencing relief from IBS symptoms and who had baseline 5-HIAA levels that fell within the predetermined range. In some methods, the TPH inhibitor responder rate for the predetermined range is the percent of IBS patients (e.g., patients in a blinded clinical study of the TPH inhibitor) who previously reported a decrease in mean stool consistency (e.g., as measured on the Bristol Stool Scale) and who had baseline 5-HIAA levels that fell within the predetermined range.

In some methods of the invention, 5-HT levels are measured by ELISA. In others, they are measured by chromatography, e.g., liquid chromatography.

In some methods of the invention, 5-HIAA levels are measured by ELISA. In others, they are measured by chromatography, e.g., liquid chromatography.

One embodiment of the invention encompasses a kit for determining whether a patient suffering from IBS will be responsive to TPH inhibitor therapy, which comprises: a device for measuring blood 5-HT concentrations; and information that correlates or provides instructions as to how to correlate measurements obtained using the device with a TPH inhibitor responder rate, wherein the TPH inhibitor responder rate is a predicted likelihood of relief from IBS symptoms for patients having a baseline 5-HT level that falls within a predetermined range. Some kits further comprises a questionnaire that can be used to determine whether the patient has IBS-D or IBS-A (e.g., whether the patient satisfies Rome III criteria). In some kits, the device utilizes an ELISA. In some kits, the TPH inhibitor responder rate for the predetermined range of 5-HT levels is the percent of IBS patients (e.g., patients in a blinded clinical study of the TPH inhibitor) who previously reported experiencing relief from IBS symptoms and who had baseline 5-HT levels that fell within that predetermined range. In some kits, the TPH inhibitor responder rate for the predetermined range of 5-HT levels is the percent of IBS patients (e.g., patients in a blinded clinical study of the TPH inhibitor) who previously reported a decrease in mean stool consistency (e.g., as measured on the Bristol Stool Scale) and who had baseline 5-HT levels that fell within that predetermined range.

Another embodiment encompasses a kit for determining whether a patient suffering from IBS will be responsive to TPH inhibitor therapy, which comprises: a device for measuring blood or urinary 5-HIAA concentrations; and information that correlates or provides instructions as to how to correlate measurements obtained using the device with a TPH inhibitor responder rate, wherein the TPH inhibitor responder rate is a predicted likelihood of relief from IBS symptoms for patients having a baseline 5-HIAA level that falls within a predetermined range. Some kits further comprises a questionnaire that can be used to determine whether the patient has IBS-D or IBS-A (e.g., whether the patient satisfies Rome III criteria). In some kits, the device utilizes an ELISA. In some, the TPH inhibitor responder rate for the predetermined range of 5-HIAA levels is the percent of IBS patients (e.g., patients in a blinded clinical study of the TPH inhibitor) who previously reported experiencing relief from IBS symptoms and who had baseline 5-HT levels that fell within that predetermined range. In some, the TPH inhibitor responder rate for the predetermined range of 5-HIAA levels is the percent of IBS patients (e.g., patients in a blinded clinical study of the TPH inhibitor) who previously reported a decrease in mean stool consistency (e.g., as measured on the Bristol Stool Scale) and who had baseline 5-HIAA levels that fell within that predetermined range.

### 6. EXAMPLES

Aspects of this invention can be understood from the following examples, which do not limit its scope.

6.1. Treatment of IBS Patients

A randomized, double-blind, placebo-controlled phase 2a clinical trial was conducted to assess the safety and efficacy of a TPH inhibitor, (S)-2-amino-3-(4-(2-amino-6-((R)-2,2,2-trifluoro-1-(3′-methoxybiphenyl-4-yl)ethoxy)pyrimidin-4-yl)phenyl)propanoic acid, in 155 patients suffering from IBS-D or IBS-A, based on Rome III criteria. Capsules containing the compound or placebo were orally administered. Two dose levels were tested: 250 mg QID and 1000 mg QID. A two-week run-in period was used to establish baseline symptoms, followed by a 28 day randomized, double-blind treatment period. There was then a two-week follow-up period.

During the study, patients' urine and blood were obtained to assess 5-HIAA and 5-HT levels. Methods used to assess clinical endpoints were: 1) global assessment by weekly questioning regarding adequate relief of IBS pain and discomfort; and 2) Bristol Stool Scale was used to assess stool consistency by daily automated telephonic IVRS contact.

It was found that patients randomized to the high dose arm showed a statistically significant improvement versus placebo in the weekly global assessment. The patients also showed significant improvement in stool consistency. These observations correlated with a reduction in plasma 5-HT levels as well as 24 hour urinary 5-HIAA levels. Even more significant was an observed correlation between the baseline serotonin levels (i.e., as determined by measuring 5-HIAA levels in urine collected over a 24 hour period prior to dosing) of patients and their improvement in global response. In particular, the drug exerted a greater beneficial effect in IBS patients who had comparatively high baseline levels than in those who did not.

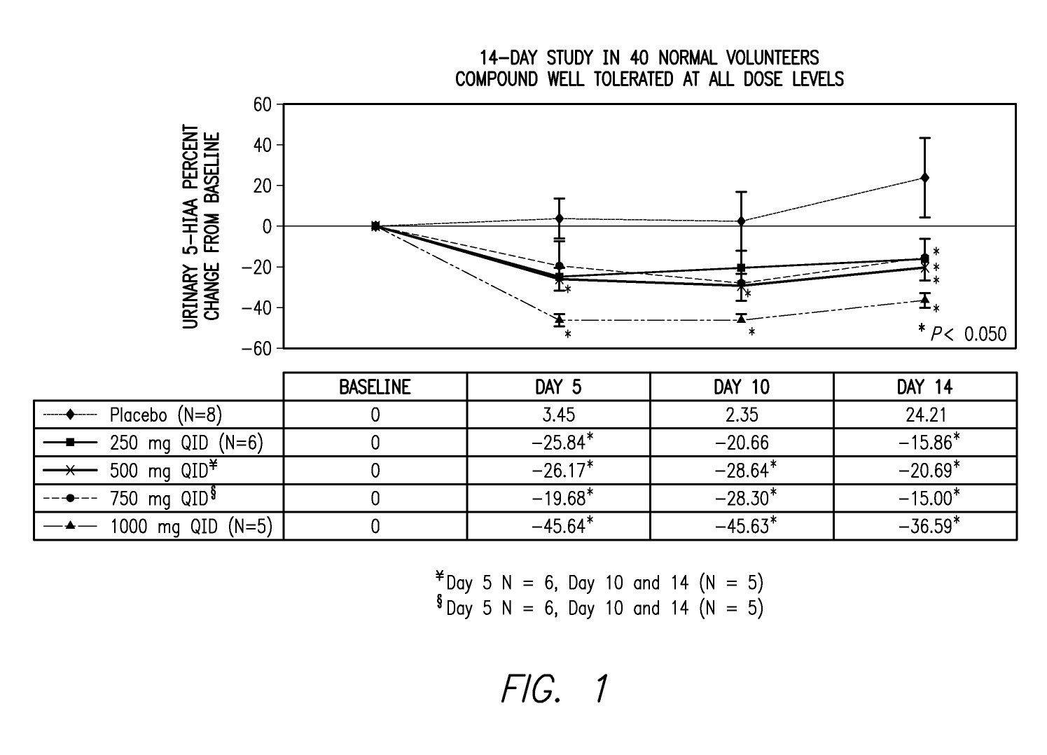

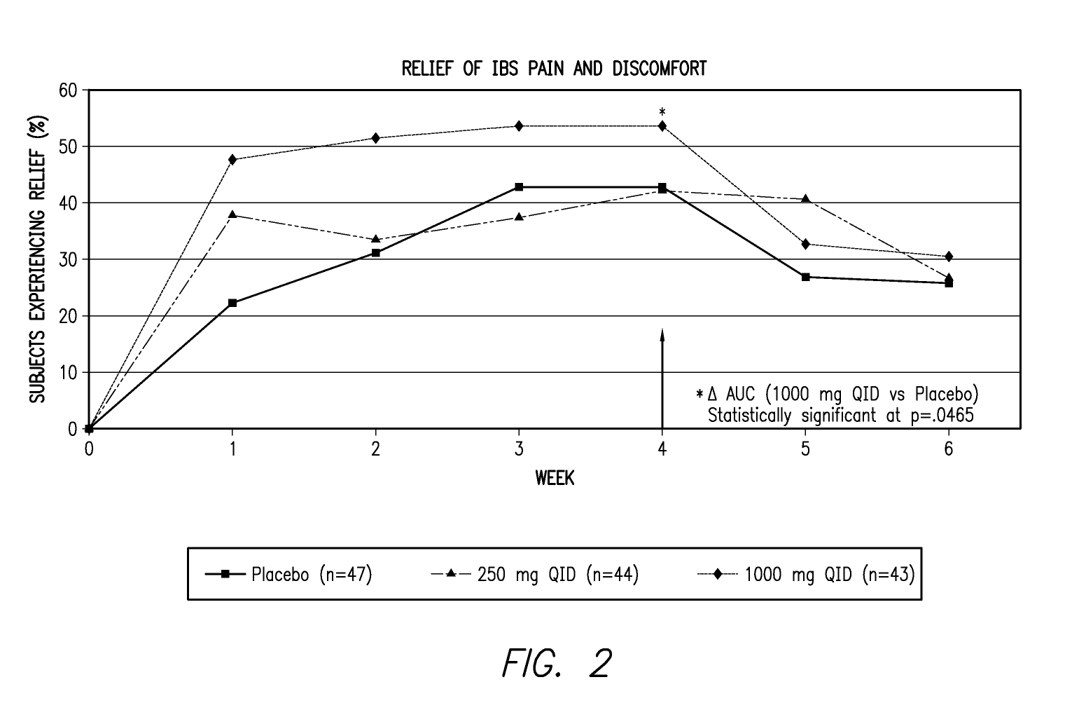

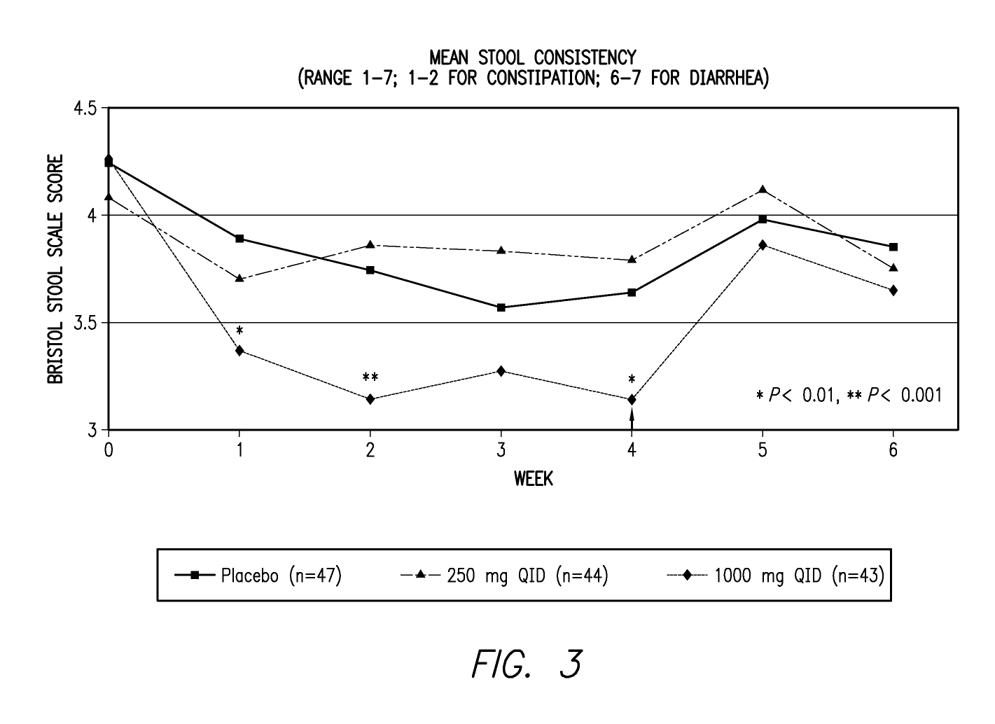

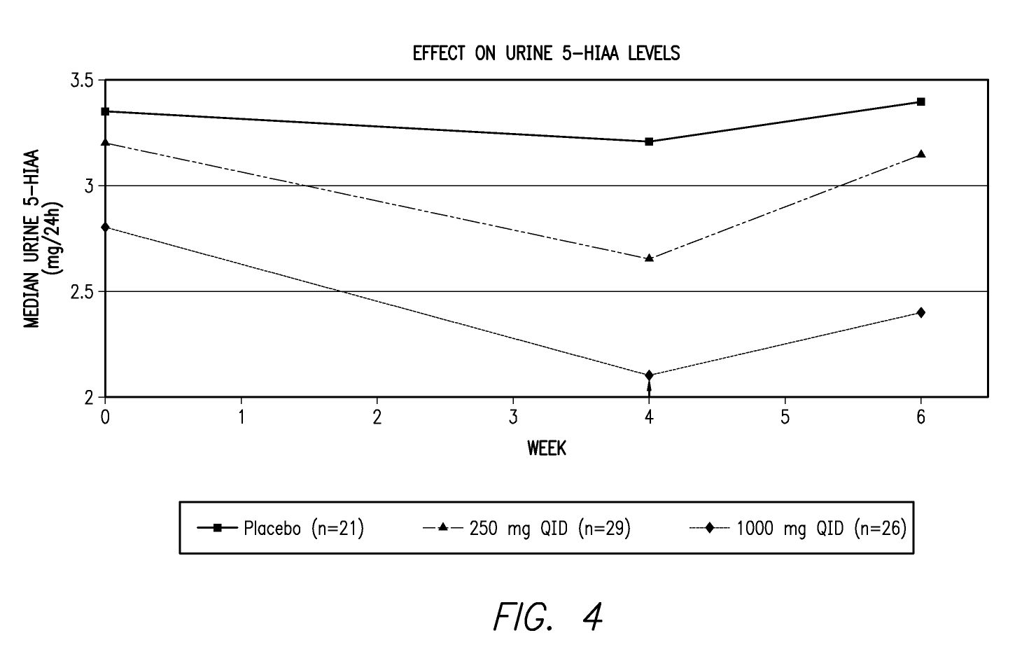

Data obtained from a phase 1b multi-dose study of (S)-2-amino-3-(4-(2-amino-6-((R)-2,2,2-trifluoro-1-(3′-methoxybiphenyl-4-yl)ethoxy)pyrimidin-4-yl)phenyl)propanoic acid in healthy normal volunteers is shown in FIG. 1. FIGS. 2-4 show data obtained from the phase 2a study of the same compound in IBS patients.

All publications (e.g., patents and patent applications) cited above are incorporated herein by reference in their entireties.

1. A method of treating or managing non-constipating irritable bowel syndrome (IBS), which comprises administering to a patient suffering from non-constipating IBS an amount of a tryptophan hydroxylase (TPH) inhibitor sufficient to lower the patient's blood 5-HT level by at least about 10 ng/mL compared to baseline.

2. (canceled)

3. The method of claim 1, wherein the 5-HT level is lowered by at least about 20 ng/mL compared to baseline.

4. The method of claim 3, wherein the 5-HT level is lowered by at least about 30 ng/mL compared to baseline.

5. A method of treating or managing non-constipating IBS, which comprises administering to a patient suffering from non-constipating IBS an amount of a TPH inhibitor sufficient to lower the patient's 5-HIAA level by at least about 10 percent compared to baseline.

6. (canceled)

7. The method of claim 5, wherein the 5-HIAA level is lowered by at least about 20 percent compared to baseline.

8. The method of claim 7, wherein the 5-HIAA level is lowered by at least about 30 percent compared to baseline.

9. The method of claim 5, wherein the 5-HIAA is urinary 5-HIAA.

10. The method of claim 9, wherein the measurement of 5-HIAA is obtained from urine collected over at least 12 hours.

11. The method of claim 5, wherein the 5-HIAA is blood 5-HIAA.

12. The method of claim 1, wherein the TPH inhibitor is administered orally.

13-20. (canceled)

21. The method of claim 5, wherein the TPH inhibitor is administered orally.
